(19) United States
(12) Patent Application Publication
(10) Pub. No.: US 2021/0262024 A1
(43) Pub. Date: Aug. 26, 2021

(54) POLYNUCLEOTIDE DUPLEX PROBE MOLECULE

(71) Applicant: UCB BIOPHARMA SRL, Brussels (BE)

(72) Inventors: Daniel John LIGHTWOOD, Slough, Berkshire (GB); Michael John WRIGHT, Slough, Berkshire (GB)

(21) Appl. No.: 17/255,316

(22) PCT Filed: Jul. 3, 2019
(86) PCT No.: PCT/EP2019/067920

§ 371 Date: Dec. 22, 2020

(30) Foreign Application Priority Data
Jul. 3, 2018 (GB) 1810901.7

(51) Int. Cl. C12Q 1/6869; C12Q 1/6806

(52) U.S. Cl. CPC C12Q 1/6869; C12Q 1/6806

(57) ABSTRACT

The present disclosure relates to a duplex probe molecule comprising: i) a double-stranded-core (the core) comprising a first polynucleotide strand (first-strand) and a second polynucleotide strand (second-strand), wherein the first and second strands are complementary to each other, ii) a single stranded first polynucleotide probe (first probe) sequence extending in the 5′ to 3′ direction from the first-strand of the core; and iii) single stranded second polynucleotide probe (second probe) sequence extending in the 5′ to 3′ direction from the second strand of the core; wherein the first and second probes extend outwards from the double stranded core in opposing directions on different polynucleotide strands and each terminate in a 3′ end. The disclosure also extends to methods of making the molecule and use of the molecule, for example to capture variable domains sequence information from antibodies.

The present disclosure relates to a method of linking two polynucleotides sequences by a barcode, which is known or unknown, which for example facilitates capture of antibody variable region cognate pairs. The disclosure also extends to the molecules prepared by the present method including intermediates, such as a double and single stranded molecule, referred to herein as a duplex probe molecule, and use of said molecules in recombinant techniques, such as capturing polynucleotide sequences for further processing.

### BACKGROUND

Capturing polynucleotides with a relationship, for example DNA or RNA encoding a VH and VL (a cognate pair) from a single antibody producing cell is of interest because in some instances the original pairings have optimised properties. So-called cognate pairs of variable regions from a single antibody or producing cell often have optimised properties, which are advantageous.

Although this can be done on a small scale by handling, for example individual B cells in discrete wells or droplets until the variable regions have been sequenced or cloned this methodology is not really conducive for high-throughput processes.

WO2013/117591 discloses a method of linking two polynucleotide sequences from a single cell by multiplex overlap PCR, preferably after performing an amplification step.

In an attempt to increase the amount of polynucleotides that can be handled WO2013/188872 discloses a process where single cells are sorted into individual compartments, such as wells. The RNA from one cell is collected on a bead with a RNA capture agent on the bead surface. Reverse transcription is performed to amplify the captured mRNA and at least two products (such as cDNA products) which have been amplified from the cell are sequenced. In some embodiments after the amplification step, the two polynucleotides are linked by performing overlapping PCR. WO2013/188872 requires many of the steps to be performed separately in an individual container for each polynucleotide, including the PCR step. Furthermore, the relationship between the linked polynucleotides is lost if the sequences are separated or contaminated early in the process.

WO2014/144495 discloses an individual compartment comprising a bead with an anchor primer and one or more barcodes attached thereto. A cell is lysed in the presence of the bead and RNA from the lysed cell is reverse transcribed onto the bead as cDNA. In some experiments the barcode is linked to RNA from the cell during transcription using a T7 promoter binding site as the unique identifier.

Complementary primers specific to the polynucleotide sequence in combination with a set of primers specific to the barcode are employed during amplification to link the barcode and the polynucleotide sequences. Thus, the relationship between the first and second polypeptide can be identified because both sequences have the same barcode attached. However, the PCR amplification must be performed in the individual wells. Furthermore, primers have to be prepared which link the barcode to the polynucleotide and therefore the sequence of the barcode needs to be known.

It would be useful to have alternative methods of linking polynucleotides and, for example appending barcode sequences, in particular, methods which are amenable to high-through-put techniques. The present disclosure employs a duplex molecular probe to simultaneously capture two polynucleotide sequences, for example mRNA from, in particular, a single cell without the need to use a solid support, such as a bead. Furthermore, the variable region encoded in the mRNA can be transcribed into the duplex probe molecule by reverse transcription. The duplex probe molecule is beneficial in that it ensures both polynucleotides have the same barcode from the outset, prior to any amplification steps. Additionally, the duplex core of the molecule can be extended employing a polymerase into a fully double stranded molecule where each strand encodes both polynucleotides physically linked via a barcode thereby providing two strands containing the same genetic information and both containing the same barcode sequence (i.e. same identity/code). Thus, the need to perform PCR to link the two polynucleotide sequences can be completely avoided, or indeed performed at a later stage.

### SUMMARY

The present disclosure is summarised in the following paragraphs:
1. A duplex probe molecule comprising:
   i) a double-stranded-core (the core) comprising a first polynucleotide strand (first-strand) and a second polynucleotide strand (second-strand), wherein the first and second strands are complementary to each other,
   ii) a single stranded first polynucleotide probe (first probe) sequence extending in the 5′ to 3′ direction from the first-strand of the core; and
   iii) single stranded second polynucleotide probe (second probe) sequence extending in the 5′ to 3′ direction from the second strand of the core;
   wherein the first and second probes extend outwards from the double stranded core in opposing directions on different polynucleotide strands and each terminate in a 3′ end.
2. A duplex probe molecule according to paragraph 1 comprising a barcoding region.
3. A duplex probe molecule according to paragraph 1 or 2 where a barcoding region is located between the first probe and the first-strand of the core.
4. A duplex probe molecule according to any one of paragraphs 1 to 3, where a barcoding region is located between the second probe and the second-strand of the core.
5. A duplex probe molecule according to any one of paragraphs 1 to 4, wherein the first-strand of the core comprises a barcode.
6. A duplex probe molecule according to any one of paragraphs 1 to 5, wherein the second-strand of the core comprises a barcode.
7. A duplex probe molecule according to any one of paragraphs 1 to 6, wherein the first strand comprises a first and third primer annealing site, for example flanking part of the first strand, such as a barcode therein.
8. A duplex probe molecule according to any one of paragraphs 1 to 7, wherein the second strand comprises second and fourth primer annealing site, for example flanking part of the second strand, such as a barcode therein.
9. A duplex probe molecule according to paragraph 1 comprising:
   a double stranded core (the core) of a first polynucleotide strand (first-strand) and a second polynucleotide strand (second-strand), wherein the first-strand comprises a barcode sequence flanked by a first and a second primer annealing site and the second-strand comprises a barcoding sequence flanked by a third and a fourth primer annealing site;
   a single stranded first polynucleotide probe sequence (first probe) extending in the 5′ to 3′ direction from the first-strand of the core; and
   a single stranded second polynucleotide probe sequence (second probe) extending in the 5′ to 3′ direction from the second-strand of the core,
   such that the first and second probes extend outwards from the double stranded core in opposing directions on different polynucleotide strands and each terminate in a 3′ end.
10. The duplex probe molecule according to paragraph 2 to 10, wherein the double stranded core comprises a barcode having 6 to 100 base pairs, such as 10 to 60, 12 to 40, 14 to 30, 16 to 20 base pairs, in particular 15 base pairs in a given polynucleotide.
11. The duplex probe molecule according to any one of paragraphs 1 to 10, wherein the first and/or second probe sequences encode one or more antibody regions, for example constant domains.
12. The duplex probe molecule according to paragraph 11, wherein the first polynucleotide probe sequence encodes a heavy chain constant region and the second polynucleotide probe sequence encodes a light chain constant region or vice versa.
13. A host cell comprising a duplex probe molecule according to any one of paragraphs 1 to 12.
14. A kit comprising one or more duplex probe molecules according to any one of paragraphs 1 to 12 and reagents and/or instructions for use.
15. A method of preparing a duplex probe molecule according to any one of paragraphs 1 to 4, comprising the steps of:
   (a) providing a strand-one comprising in the 3′ to 5′ direction, a first polynucleotide probe, a first primer annealing site, a barcoding sequence and a restriction site, and:
      i. annealing to strand-one a first primer specific to the first primer annealing site, and
      ii. employing a polymerase to synthesise the complementary polynucleotide sequence from the first primer along the length of strand-one in the 5′ to 3′ direction to provide a double stranded barcode region, and
   (b) providing a strand-two comprising in the 3′ to 5′ direction, the second polynucleotide probe, a second primer annealing site, a barcoding sequence and a restriction site, and annealing to strand-two:
      i. a second primer specific to the second primer annealing site, and
      ii. employing a polymerase to synthesise the complementary polynucleotide sequence from the second primer along the length of strand-two in the 5′ to 3′ direction to provide a double stranded barcode region and,
   (c) cutting a double stranded part of strand-one and strand-two with a restriction enzyme specific to the restriction site encoded therein, in the same or separate reactions; and
   (d) ligating the sticky ends of strand-one and strand-two obtained from step c) to form a duplex probe molecule comprising a double stranded core made up of the double stranded region from strand-one ligated to the double stranded 5′ region from strand-two, such that the first probe sequences and second probe sequence each extend as a single strand from the relevant double stranded region in opposing directions such that each single strand terminates in a 3′ end.
16. The method according paragraph 15, wherein the sticky ends have a sense strand that is non-palindromic to a sticky end in the corresponding antisense strand of polynucleotide.
17. The method according to paragraph 15 or 16 wherein the restriction site is one that is cut using an enzyme selected from the group consisting of: AciI, AcuI, AlwI, BaeI, BbsI, BbvCI, BbvI, BccI, BceAI, BcgI, BciVI, BcoDI, BfuAI, BmrI, BpmI, Bpul0I, BpuEI, BsaI(1), BsaI-HF®, BsaXI, BseRI, BseYI, BsgI, BsmAI, BsmBI, BsmFI, BsmI, BspCNI, BspMI, BspQI, BsrDI, BsrI, BssSGI, BssSI, BtgZI, BtsaI, BtsCI, BtsI, BtsI MutI, CspCI, Earl, EciI, EciI, EcoP15I, FauI, FokI, FspEI, HgaI, HphI, HpvAV, I-CeuI, I-SceI, LpnPI, MboII, MmeI, MnlI, MspJI, Nb.BbvCI, Nb. BsmII, Nb.BsrDI, Nb.BtsI, NmeAIII, Nt.AlwI, Nt.BbvCI, Nt.BsmAI, Nt.BspQI, Nt.CviPII, PI-PspI, PI-SceI, PleI, SapI and SfaNI.
18. The method according to paragraph 17, wherein the restriction enzyme is BbvCI, in strand-one and/or strand-two.
19. The method according to any one of paragraphs 15 to 18, wherein the same restriction enzyme is used to cut both strand one and strand two.
20. The method according to any one of paragraphs 15 to 19, wherein the strand-one and/or strand-two molecules further comprise one or more base pairs upstream (in the 5′ direction) of the restriction site, such as 1, 2, 3, 4, 5, 6 or more base pairs.
21. The method according to any one of paragraphs 15 to 20, wherein the barcode comprises 3 to 50 base pairs, such as 5 to 20, 6 to 20, 7 to 15, 8 to 10 base pairs, in particular 15 base pairs.
22. A method of making a duplex probe molecule, said method comprising the step of annealing a polynucleotide molecule-one (molecule-one) comprising in the 3′ to 5′ direction a first polynucleotide probe (first probe) and a first polynucleotide strand (first-strand), with a polynucleotide molecule-two (molecule-two) comprising in the 3′ to 5′ direction a second polynucleotide probe (second probe) and second polynucleotide strand (second-strand), wherein the first-strand and second-strand are complementary and the first probe and second probe are not complementary and annealing the molecule-one and two provides a duplex probe molecule.
23. A method of covalently capturing a first and/or second polynucleotide sequence of interest (such as mRNA), comprising the steps of:
   a) hybridising each polynucleotide sequence to its complementary probe sequences in the single stranded portions of a duplex probe molecule as defined in any one of claims 1 to 12; and
   b) extending the 3′ end of the first and second polynucleotide probe sequences by synthesising the cDNA complementary to the polynucleotide sequences hybridised to the probe sequences, for example employing a reverse transcriptase.
24. The method according to paragraph 23, further comprising the step of c) synthesising the DNA sequence complementary to the newly synthesised cDNA, for example by employing a polymerase, so as to provide a fully double stranded duplex molecule.
25. The method according to paragraphs 23 or 25, wherein prior to step a) the duplex probe molecule as defined in any one of paragraphs 1 to 4 is transfected into a target cell.
26. The method according to paragraph 25, which comprises a further step of lysing the target cell to recover the duplex probe molecule with the first and/or second polynucleotide sequences of interest hybridised thereto.
27. The method according to paragraph 25 or 26, wherein a plurality of different duplex molecules are transfected into the target cell.
28. The method according to any one of paragraphs 24 to 28, wherein the DNA sequence complementary to the newly synthesised cDNA is synthesised by reverse transcription using primers complementary to the leader sequence(s) of the hybridised polynucleotide sequences of interest.
29. A method according to any one of paragraphs 23 to 28 which comprises a PCR step to amplify part or all of a DNA sequence is the duplex probe molecule.
30. The method according to paragraphs 23 to 29, further comprising the step of:
   a) sequencing part or all of one or more strands of duplex molecules in order to determine the sequence of the newly synthesised cDNA.
In an independent aspect the present disclosure also provides a duplex probe molecule comprising:
   a double stranded core (the core) formed by a first and a second polynucleotide strand, wherein each strand comprises a barcode sequence flanked by a first and a second primer annealing site; a single stranded first polynucleotide probe sequence extending in the 5′ to 3′ direction from the first primer annealing site in the first polynucleotide strand of the core; and
   a single stranded second polynucleotide probe sequence extending in the 5′ to 3′ direction from the second primer annealing site in the second polynucleotide strand of the core,
   such that the first and second polynucleotide probes extend outwards from the double stranded core in opposing directions on different polynucleotide strands and each terminate in a 3′ end.
Alternatively, the duplex probe molecule can be described as comprising:
   a first strand (strand-one) which sequentially from its 3′ end has a first polynucleotide probe sequence, a first primer annealing site, a barcoding region and a second primer annealing site; and
   a second strand (strand-two) which sequentially from its 3′ end has a second polynucleotide probe sequence, a second primer annealing site, a barcoding region and a first primer annealing site,
      wherein the following sites in each strand are paired to form a double stranded core, first annealing site, second annealing site and barcoding region.

Generally, the barcoding region on strand-one and strand-two are complementary and are the same barcode in each duplex probe molecules of the present disclosure.

Generally, the first primer annealing site will be complimentary in strand-one and strand-two of each duplex probe molecule of the present disclosure.

Generally, the second primer annealing site will be complementary in strand-one and strand-two of each duplex probe molecules of the present disclosure.

The duplex probe molecule may further comprise one or two cDNA sequences captured by extending a polynucleotide probe sequence or the polynucleotide probe sequences, for example by employing reverse transcription, such as described herein.

In one embodiment the double stranded core comprises a barcode having 6 to 100 base pairs, such as 10 to 60, 12 to 40, 14 to 30, 16 to 20 base pairs, in particular 30 base pairs in total in the two strands i.e. 15 bases in each strand where the sequences are complementary to each other.

In one embodiment the first single stranded polynucleotide probe sequence anneals to a polynucleotide sequence encoding an antibody chain, for example an antibody heavy chain or an antibody light chain, in particular the first single stranded polynucleotide probe anneals to a polynucleotide sequence (referred to herein as a target sequence), such as an mRNA sequence, through a region in the probe which is complementary to the target sequence.

In one embodiment the second single stranded polynucleotide probe sequence anneals to a polynucleotide sequence encoding an antibody chain, for example an antibody heavy chain or an antibody light chain, in particular the second single stranded polynucleotide probe anneals to a polynucleotide sequence (referred to herein as a target sequence), such as an mRNA sequence, through a region in the probe which is complementary to the target sequence.

In one embodiment the first polynucleotide probe sequence encodes at least part of a heavy chain constant region and the second polynucleotide probe sequence encodes at least part of a light chain constant region. In one embodiment the first polynucleotide probe sequence encodes at least part of a light chain constant region and the second polynucleotide probe sequence encodes at least part of a heavy chain constant region.

The present disclosure also provides a method of preparing a duplex probe molecule according to the present disclosure comprising the steps of:
(a) providing a strand-one comprising in the 3′ to 5′ direction: a first polynucleotide probe (also referred to herein as a first single stranded polynucleotide probe sequence), a first primer annealing site, a barcoding sequence and a restriction site suitable for cutting with an appropriate restriction enzyme, and:
   i. annealing to strand-one a first primer specific to the first primer annealing site, and
   ii. employing a polymerase (such as a DNA polymerase) to synthesise the complementary polynucleotide sequence from the first primer along the length of strand-one in the 5′ to 3′ direction to provide a double stranded barcode region (for example as shown on FIG. 2 or 3A); and
(b) providing a strand-two comprising in the 3′ to 5′ direction: a second polynucleotide probe, a second primer annealing site, a barcoding sequence and a restriction site suitable for cutting with an appropriate restriction enzyme, and
   i. annealing to strand-two a second primer specific to the second primer annealing site, and
   ii. employing a polymerase (such as a DNA polymerase) to synthesise the complementary polynucleotide sequence from the second primer along the length of strand-two in the 5′ to 3′ direction to provide a double stranded barcode region and,
(c) providing sticky ends by cutting a double stranded part of strand-one (from step a)ii) and strand-two (from b)ii)) with a restriction enzyme specific to the restriction site encoded therein, in the same or separate reactions; and
(d) ligating the sticky ends of strand-one and strand-two obtained from step c) to form a duplex probe molecule comprising a double stranded core made up of the double stranded region from strand-one ligated to the double stranded region from strand-two, such that the first polynucleotide probe sequences and second polynucleotide probe sequence each extend as a single strand from the relevant double stranded region in opposing directions and each single stranded probe sequence terminates in a 3′ end.

In one embodiment a restriction enzyme is employed to cut a double stranded section of strand-one and/or strand-two at a temperature in the range 25 to 40° C., for example 30 to 37° C., such as 30° C.

In one embodiment a polymerase (such as a DNA polymerase) in step a) part ii) and/or in step b) part ii) is employed at a temperature in the range 20 to 37° C., for example 25° C.

In one embodiment one or more steps of the method according to the present disclosure are performed at 40° C. or less, for example ambient temperature, such as about 20° C. Advantageously performing part or all of the method of the present disclosure in said temperature range helps to ensure the target polynucleotide(s) stay associated with the duplex probe molecule until the genetic information therefrom is captured.

In one embodiment the method comprises the further step of using the duplex probe molecule to hybridise a polynucleotide of interest thereto, for example an mRNA sequence to the first and/or second polynucleotide probe sequences of said duplex probe molecule according to the present disclosure, in particular the first and second polynucleotide probe sequences concomitantly anneal to the polynucleotide (such as mRNA), which they respectively recognise.

In one embodiment the method comprises synthesising onto the 3′ end of the first and/or second polynucleotide probe sequence cDNA complementary to the mRNA hybridised thereto, for example employing a reverse transcriptase.

In one embodiment there is no PCR step involved in covalently connecting a barcode to the captured polynucleotide.

In one embodiment there is provided use of a duplex probe molecule according to the present disclosure to capture at least one, such as two polynucleotide sequences of interest, for example from a cell, by hybridising thereto.

### DETAILED DISCLOSURE

Duplex probe molecule as employed herein refers to a single-stranded, double-stranded, single-stranded molecule with two 3′ ends as described herein, for example as illustrated diagrammatically in FIG. 1 (before addition of a polynucleotide of interest from the cell) and FIG. 3A (after addition of polynucleotides of interest from the cell). The single-stranded, double-stranded, single-stranded molecules are also referred to as ss/ds/ss.

Strand-one (or first strand) as employed herein is an arbitrary identifier of a polynucleotide (such as a DNA sequence) that is or ultimately becomes a component of the duplex probe molecule of the disclosure. At early stages of the process it may be single stranded and later in the process it is single stranded along part of its length and double stranded along part of its length or the remainder of its length i.e. going from a single stranded sequence to a single and double stranded sequence is illustrated in FIG. 4A-C.

Strand-two (or second strand) as employed herein is an arbitrary identifier of the “second” or “other” polynucleotide that is or becomes part of the duplex probe molecule of the present disclosure. As discussed above for strand-one, at early stages of the process it may be single stranded and later in the process it is single stranded along part of its length and double stranded along part of its length or the remainder of its length i.e. going from a single stranded sequence to a single and double stranded sequence is illustrated in FIG. 4A-C.

Strand-one and strand-two can be prepared by methods known in the art, for example independently selected from techniques including but not limited to synthesis of part or all of the sequence, recombinant techniques and one or more ligation steps. In one embodiment at least the barcoding section in strand-one and/or strand-two is synthesised.

A polynucleotide probe is a polynucleotide sequence, for example RNA or DNA, in particular a DNA sequence, which can be employed to hybridise to a polynucleotide sequence of interest, for example from a cell (i.e. the polynucleotide probe sequence is complementary to at least part of a sequence in a polynucleotide of interest, such as complementary to an mRNA sequence, for example produced by a cell). The method and molecules of the disclosure comprise a first and second polynucleotide probe, and generally the two probes are complementary to different target polynucleotide sequences.

In one embodiment a polynucleotide probe sequence (i.e. one or both) employed in the duplex probe molecule of the present disclosure is at least 25 nucleotides in length, such as 26, 27, 28, 29, 30 or more nucleotides in length.

The skilled person knows how to design probe molecules to anneal to a polynucleotide of interest and generic sequences are available to capture a polynucleotide of unknown sequence, for example polynucleotides probe sequence may be selected from the group comprising a poly(T), a sequence specific for at least part of an antibody heavy chain, such as part of a constant region/domain, a sequence specific for at least part of an antibody light chain, such as part of a constant region/domain. Poly(T) polynucleotides capture poly(A) mRNA tails and may be employed in a duplex probe of the present disclosure.

As mentioned the first and second probe sequences are generally directed to different polynucleotides of interest. In one embodiment the first probe is a sequence complementary to at least part of a heavy chain constant region, for example complementary to the polynucleotide sequence encoding CH1 or a fragment thereof, and the second probe sequence may encode at least part of a light chain constant region, for example complementary to a polynucleotide sequence encoding CKappa (or CLambda) or a fragment thereof, or vice versa the first probe may encode at least part of a light chain constant region (such as CKappa or CLambda or a fragment thereof) and the second probe may encode at least part of a heavy chain constant region (such as CH1 or a fragment thereof).

Alternatively, or additionally the polynucleotide probe may be complementary to a J region in an antibody or a framework region in an antibody.

Advantageously, the polynucleotide probes in the duplex probe molecule of the present disclosure hybridise to a relevant portion of polynucleotide of interest from a cell, such as mRNA and “capture” the same. The first polynucleotide probe sequence may capture mRNA for an antibody heavy sequence and the second polynucleotide probe sequence may capture mRNA for an antibody light sequence or vice versa. Whilst, for example the mRNA is held by a polynucleotide probe sequence the mRNA can be employed as a template to synthesise the sequence encoding the antibody variable region from the relevant antibody chain onto the 3′end of that polynucleotide probe. The synthesis will generally continue through to the leader sequence of the mRNA and then terminate. Thus, employing a reverse transcriptase enzyme allows the information in the captured mRNA to be transcribed onto the 3′ end of the relevant polynucleotide probe sequence and in the duplex probe molecule of the present disclosure. Therefore, between the first and second polynucleotide probes sequences in the duplex molecules of the present disclosure sequence encoding antibody variable regions as a cognate pairs can be captured.

In one embodiment the two target polynucleotides are “captured” concomitantly. Captured concomitantly as employed herein refers to the polynucleotides being captured at “approximately the same time”, as opposed to being captured at two distinct time points.

The polynucleotide probe sequences employed in the methods and molecules of the present disclosure need to be an adequate length to hybridise efficiently to the polynucleotide of interest from the cell. Polynucleotide probe sequences in the range of 10 to 40 bases are generally suitable for the use in the present disclosure.

The polynucleotide probe sequence employed does not require perfect complementarity to the polynucleotide of interest, such as mRNA from the cell. If the probe is fit for the purpose of hybridising to an mRNA molecule of interest from the cell then the probe is suitable for use in the duplex probe molecule of the present disclosure.

In one embodiment the probe sequence has at least 90% identity or similarity to the target sequence, for example 91, 92, 93, 94, 95, 96, 97, 98, 99 or 100% identity or similarity to the target sequence.

First and second polynucleotide probes extending outwards from the double stranded core as employed herein is illustrated in FIG. 1, FIGS. 5A-C shows how the probes act to capture polynucleotide sequences of interest.

The term ‘polynucleotide recognised’, ‘polynucleotide of interest’, ‘target polynucleotide’ are employed interchangeably herein unless the context indicates otherwise, to refer to a polynucleotide with genetic information which it is desirable to capture in the duplex probe molecule of the present disclosure.

The term “barcode”, “barcode region”, “barcode sequence”, “barcoding region” or “barcoding sequence” as used herein refers to a nucleotide sequence (also referred to as a polynucleotide sequence) which serves as a unique identifier. Polynucleotide barcodes (also referred to as DNA barcodes) are generally random (in particular non-coding) combinations of guanine (G), adenine (A), thymine (T) or cytosine (C). A base which represents any one of G, A, T or C is given the designation N herein.

Generally, the greater the number of nucleotides used the greater the potential complexity of the barcode, which in turn may increase the number of unique barcodes available for use. For example, using 4 of each of the bases i.e. 4×4×4×4 provides 256 unique barcodes. If the barcode is used to link one or more known polynucleotide sequences, care should be taken when designing the barcode to ensure that the barcode has a different sequence from the polynucleotide sequences of interest and thus can be readily differentiated from the polynucleotide sequences. However, generally the barcode employed will be randomised. Accordingly, the barcode may be, for example non-coding, i.e. does not encode a polypeptide or protein.

Alternatively, the position of the barcode in the sequence may be defined very precisely so that its exact location in the sequence of strand-one and/or strand-two is known/allocated.

It is also advisable to have a barcoding sequence which is different/differentiated from the first and second primer annealing sites to ensure that the primers employed can specifically hybridise to the primer annealing site.

The barcoding sequence may be read by sequencing techniques to reveal the code, in particular next generation sequencing techniques.

In one embodiment the barcode is a simple barcode i.e. just a unique identifier.

The barcodes may be designed such that there is a code within a code, for example the presence of a particular fragment of the same or similar barcode on multiple sequences may indicate that a common feature(s) is shared between the multiple sequences. In one embodiment, for example, part of the barcode for various duplex probe molecules may be the same to represent the molecules having a common first polynucleotide probe sequence and the remainder of the barcode may be unique to represent variable/different second polynucleotides probe sequences. This may be useful, for example for screening variant sequences that may function well in combination together or have substantially similar function. An approach such as this may be used in a library of duplex probe molecules. Another example may be barcodes with a certain fragment-code therein may indicate a certain isotype such as IgG is captured by the polynucleotide strand.

The barcodes employed in the molecules and methods of the present disclosure may be known or unknown. In one embodiment the barcoding sequence is unknown including partially unknown or fully unknown. Unknown barcodes, in particular may be sequenced, for example employing next generation sequencing techniques to reveal the unique identifier code. The barcodes may be sequenced together with the attached polynucleotide of interest, thereby allowing the barcode and its linked polynucleotide or polynucleotides to be identified. The actual sequence of the “top” and “bottom” strand of the double stranded DNA barcode will actually be different and will generally be 100% complementarity, which allows them to be co-identified. AAAAAT is ATTTTTT etc.

Incorporating a single (distinct) barcode for each pair of “linked” polynucleotides allows for the pooling and parallel processing of the polynucleotides, without loss of the original relationship. This may be particularly useful for high throughput sequencing applications, or similar.

In one embodiment, the barcode comprises 3 to 50 bases, for example 5 to 20, 6 to 20, 7 to 15, 8 to 10, in particular 15 bases in each strand of the duplex probe molecule. In general, the greater the number of bases used, the greater the number of possible permutations and hence the greater the number of unique barcodes available for use. 30 bases in the barcode in a single strand provides about 1.53×10¹⁸ possible permutations.

Bases refers to nucleobases i.e. A, T, C, G and U. In one embodiment bases refers to DNA bases i.e. A, T, C and G. Nucleotides refers to DNA bases unless the context indicates otherwise.

Base pairs as employed herein refers to the complementary relationship between nucleobases i.e. A with T, C with G and A with U. In one embodiment base pairs refers to DNA base pairs i.e. A with T and C with G.

Advantageously, about 15 bases in one strand strikes a good balance between providing more than one million unique barcodes and lowering manufacturing costs by reducing the number of base pairs used in the barcode.

In one embodiment the barcode flanks the central region (core) of the molecule.

In one embodiment the barcode is contained with the central region (core) of the molecule.

A primer annealing site as employed herein refers to a site in a polynucleotide sequence which is recognised by a primer i.e. a location where the primer anneals (hybridises/attaches to). This can also be described as a site to which the primer is complementary. Thus, generally first and second is nominal nomenclature to indicate they are different sites in different locations. First primer annealing site and second primer annealing site can be understood in the context in which they are used, in particular, the first primer annealing site is sandwiched between a first polynucleotide probe sequence and a barcoding sequence and the second primer annealing site is located between a second polynucleotide probe sequence and a barcoding region.

At some stages of the method. the first primer annealing site and second primer annealing site are on strand-one and strand-two respectively. In the “final” duplex probe molecule of the disclosure (with single stranded polynucleotide probes extending from the double stranded core) the first and second primer annealing sites are each (both) provided in strand-one and also in strand-two and the two strands together form the double stranded sequence flanking the double stranded-barcoding sequence.

‘Primer region’ and ‘primer site’ are employed interchangeably herein with ‘primer annealing site’. Thus, the term “primer region” or “priming site” as used herein therefore refers to a sequence on a polynucleotide strand, which is complementary to a primer such that the primer binds to it or recognises it.

The term ‘primer annealing site’ is employed throughout the present specification for regions in single stranded sequences but is also used to refer to said region even after it becomes part of a double stranded section of polynucleotide sequence. Of course, when it is part of a double stranded polynucleotide sequence a primer annealing site cannot perform the function of annealing to a primer unless the two strands are separated.

Annealing in the context of the present disclosure refers to sticking to/recognising/hybridising to a relevant sequence. Annealing is enabled by complementarity in the sequences in question. The language is not intended to infer anything about the conditions under which the annealing takes place.

The primer annealing site (be it the first or second primer annealing site) is important because addition/hybridisation of the relevant primer is the starting point for synthesising the duplex double stranded core.

FIG. 9 illustrates a method where a double stranded barcoding section (also referred to herein as barcoding region) is synthesised by annealing a first primer to the first primer annealing site followed by employing a polymerase (such as DNA polymerase to synthesise a complementary strand) along the barcoding section to form a double stranded section/region. A corresponding molecule is prepared for strand-two employing similar (analogous) methods described herein for strand-one. The double stranded barcoding sections in each “strand” also comprises a restriction site after the 5′ end of the barcode.

Double stranded barcoding region (also referred to herein as barcoding section) as employed herein refers to a complementary double stranded section comprising at least the barcode.

The term “primer” as used herein refers to a short, single-stranded polynucleotide sequence, such as a short DNA sequence, typically 10-40 bases long, which anneals to its complementary sequence (“priming site” or primer region) in strand-one or strand-two, as appropriate and allows a polymerase to initiate replication. Generally, the identity of the primer sequence or sequences is known because, for example the primer or primers may have been specifically designed.

FIG. 5A-C shows a diagrammatic representation of a primer hybridising to a primer annealing site followed by extension/synthesis of a double stranded barcoding region.

In one embodiment the primer is in the region of about 16 to 30 bases. In some embodiments, the primers consist of at least 16, 17, 18, 19, 20, 21, 22, 23, 24, 26, 28, 30 or more nucleotides. Non-limiting examples of commonly used universal primers can be found in, for example, Messing (2001) Methods MoI. Biol. 167:13-31; and in Alphey, DNA Sequencing (Introduction to BioTechniques), p. 28, Garland Science; 1st edition (1997).

Any number of other suitable primers can be designed by one of skill in the art, using for example, PROBEWIZ software available at www.cbs.dtu.dk/services/DNAarray/probewiz.php or other tools. In some embodiments, the primers are selected from the primers listed in SEQ ID NO: 4 CGCAGGGCGCAGCTCGGAC and SEQ ID NO: 3 GGGACGCGCCCGTGTGCAG and their complementary sequences.

Other examples of suitable primers for use in the present invention are provided in SEQ ID NO:15, SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 and SEQ ID NO:28. In some embodiments the primers are selected from the primers listed in SEQ ID NO:15, SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 and SEQ ID NO:28.

Primers of the present disclosure are designed to be complementary to the primer annealing site in strand-one or strand-two. The primer is generally complementary over its whole length to the primer annealing site in strand-one or strand-two. However, the primer annealing site in strand-one or strand-two only represents a small section or proportion of said strand.

Accordingly, unless the context indicates otherwise the primers must be “substantially complementary” to their corresponding priming sites as employed herein means that the primers are sufficiently complementary to their priming sites to hybridize.

Thus, complementarity between the primer and primer annealing site need not to be perfect; there may be any number of base pair mismatches. However, if the number of mismatches is so great that no hybridization can occur then the primer/priming annealing site the primer is not viable. In one embodiment the probe is about 95% similar or identical over the relevant length, for example 96, 97, 98, 99 or 100% similar or identical to the relevant primer annealing site.

In general, primers of the present disclosure should be designed to minimize self-hybridization to avoid hairpin structures and cross-hybridization with both each other and other components of the reaction mixtures.

In addition, the primers designed (and if appropriate barcodes) may be compared to the known sequences to avoid hybridization to probe sequences, and/or barcoding sequences and thereby ensure that the primer is specific for the relevant primer annealing site. For example, a BLAST search can be performed for the primers (and for example barcodes) against known human sequences, e.g., at www.ncbi.nlm.nih.gov. There are numerous other algorithms that can be used for comparing and analysing polynucleotide sequences which will be known to the skilled addressee.

Advantageously the method of the present disclosure allows the use of the same primer sequences on multiple occasions and avoids the need to design new primers each time the method is performed.

After the primer or primers are annealed a DNA polymerase is employed to extend the “strand” into a double stranded section as shown in FIG. 5A-C. This extending is also referred to herein as synthesising. Suitable polymerases include those known to persons skilled in the art, for example a DNA polymerase that works at temperatures which do not disrupt the annealing of the primer(s), such as Sulfolobus polymerase, Mako polymerase, polymerase, Klenow exo neg DNA polymerase or the like.

After synthesis of a double stranded barcoding section the restriction site strand is cut with an appropriate restriction enzyme.

Generally, these restriction sites will be cut to leave sticky (or overhanging) ends and then ultimately the two sticky ends are ligated to provide a duplex probe molecule of the present disclosure. In one embodiment strand-one and strand-two are prepared separately, for example in different containers for steps such as the polymerase step and/or the restriction enzyme step. In one embodiment one or both of these steps are concomitantly performed in the same container for strand-one and strand-two, in particular when the restriction site encoded in strand-one and strand-two are the same. That is both strand-one and strand-two can be cut in the same reaction by a single type of restriction enzyme.

In one embodiment a restriction site in strand-one is the same as the restriction site in strand-two.

Sticky ends as employed herein refers to where cutting a double stranded polynucleotide sequence, such as double stranded DNA leaves one strand with an overhang of at least one base.

In one embodiment, the sticky ends have a sense strand that is non-palindromic to a sticky end in the corresponding antisense strand of polynucleotide. Advantageously, the use of non-palindromic restriction sites favours the formation of strand one/strand two duplex molecules and reduces the likelihood of undesirable strand one/strand one or strand two/strand two duplexes forming during the ligation step.

In one embodiment, the restriction site is one cut by an enzyme selected from the group comprising or consisting of: AciI, AcuI, AlwI, BaeI, BbsI, BbvCI, BbvI, BccI, BceAI, BcgI, BciVI, BcoDI, BfuAI, BmrI, BpmI, Bpul0I, BpuEI, BsaI(1), BsaI-HF®, BsaXI, BseRI, BseYI, BsgI, BsmAI, BsmBI, BsmFI, BsmI, BspCNI, BspMI, BspQI, BsrDI, BsrI, BssSαI, BssSI, BtgZI, BtsaI, BtsCI, BtsI, BtsI MutI, CspCI, Earl, EciI, EciI, EcoP15I, FauI, FokI, FspEI, HgaI, HphI, HpvAV, I-CeuI, I-SceI, LpnPI, MboII, MmeI, MnlI, MspJI, Nb.BbvCI, Nb. BsmII, Nb.BsrDI, Nb.BtsI, NmeAIII, Nt.AlwI, Nt.BbvCI, Nt.BsmAI, Nt.BspQI, Nt.CviPII, PI-PspI, PI-SceI, PleI, SapI and SfaNI.

Advantageously, these restriction enzymes recognise sites that are non-palindromic and after cutting also produce sticky ends. The production of sticky ends has the benefit of enhancing the efficiency of the ligation step because sticky ends are typically more efficiently ligated together than blunt ends.

In one embodiment, the restriction sites in both strand-one and strand-two are cut by the same restriction enzyme. Advantageously, using the same restriction enzyme simplifies the process. In one embodiment, the restriction site is cut by the enzyme BbvCI.

In an alternative embodiment, the restriction sites in both strand-one and strand-two are cut by different restriction enzymes, for example independently selected from a restriction enzyme disclosed herein. Different restriction enzymes can be used provided that when cut with the enzymes, the sticky ends in strands one and two are compatible with each other and can be ligated together.

Thus, in one embodiment the restriction sites in strand-one and strand-two are the same and both strands are cut by the same enzyme, for example in a one-pot reaction.

In one embodiment the restriction enzyme site in strand-one and strand-two are cut by different restriction enzymes. However, because the restriction enzymes are specific to a given site, two enzymes may be employed in a one-pot reaction mixture comprising strand-one, strand-two and both restriction enzymes.

Unless the context indicates otherwise one-pot reaction and one-pot reaction mixture as employed herein refers to where the relevant reaction on reactions occur in one container with both strand-one and strand-two present.

In one embodiment strand-one and/or strand-two further comprise one or more base pairs upstream and/or downstream as appropriate of the restriction site, such as 1, 2, 3, 4, 5, 6 or more base pairs. In one embodiment, 6 or more base pairs are added upstream of the restriction site sequence, wherein upstream refers to the direction moving away from the polynucleotide probe and barcoding sequence. Generally, these base pairs will not encode functional RNA nor polypeptides. Thus in one embodiment the base pairs may be referred to as “junk DNA”. Advantageously, these base pairs may help the restriction enzymes to cleave their restriction sites more efficiently.

In one embodiment one of the final steps in the formations of the duplex molecule of the disclosure is a ligation employing a ligase, such as T4 ligase to form the double stranded core section by forming a covalent phosphodiester bond between two DNA strands, for example from sticky ends left by the cutting stand-one and two with appropriate restriction enzymes.

Other suitable ligases include T3 ligase, T7 ligase and Taq ligase.

This joins the double stranded region/section in “strand-one fragment” to the double stranded region/section in “strand-two fragment”.

Strand-one fragment as employed herein refers to strand-one and the partial second strand associated therewith and forming the double stranded barcoding region therein.

Strand-two fragment as employed herein refers to strand-two and the partial second strand associated therewith and forming the double stranded barcoding region therein.

The duplex probe molecules of the present disclosure are employed to capture polynucleotides of interest from, for example a cell and copies of the captured sequence are synthesised onto the polynucleotide probes. The latter may be achieved, for example employing mRNA captured by the probe as a template and a reverse transcriptase.

The present method avoids the need to perform individual PCR steps to connect the two polynucleotides of interest, in a so-called ‘pull-through’ PCR. Thus, in one embodiment the method does not comprise a pull-through PCR step. This also avoids the requirement to know the sequence of the barcode and allows two polynucleotide sequences to be linked efficiently and simply.

Alternatively, PCR may be employed as an amplification step.

In one embodiment the linking is conceptual as opposed to purely physical as both polynucleotides have the same barcoding sequence. Thus, the strands from the molecule in FIG. 3A may be separated and yet the relationship between them can still be identified because both single strands comprise the same barcode.

In one embodiment the linking is physical, for example covalent.

In one embodiment the linking is non-physical, for example non-covalent.

In one embodiment the linking is physical, for example one DNA strand comprises both polynucleotides separated by a single stranded barcode sequence, in particular where both DNA sequences are linked to a single barcode covalently bonded thereto. For example, when the molecule is prepared in the fully double stranded form then the information for both captured mRNA sequences are encoded in each DNA strand in the molecule and thus the polynucleotides “captured” are physically connected.

This physical linking can be achieved by using a polymerase, such as a DNA polymerase to make the duplex probe molecule fully double stranded along its whole length.

The duplex probe molecule or molecules of the present disclosure may be introduced into a droplet, for example using microfluidics.

Also provided is a method comprising the step of hybridising to a duplex probe molecule of the present disclosure to a polynucleotide molecule, such as an RNA molecule from a cell (for example from cell lysate) in particular with the first polynucleotide probe and/or the second polynucleotide probe, in said duplex probe molecule.

Thus, in one embodiment the method of the present disclosure further comprises the step of:
extending the first probe sequence to synthesise cDNA corresponding to polynucleotide molecule hybridised thereto, such as RNA hybridised thereto (in particular, to capture a variable region from an antibody), and
extending the second polynucleotide probe to synthesis cDNA corresponding to polynucleotide molecule hybridised thereto, such as RNA hybridised thereto (in particular, to capture a variable region from an antibody),
thereby capturing a first and second polynucleotide sequences of interest, for example from a cell (in particular a cognate pair of antibody variable regions from a single cell).

At this stage the first and second polynucleotides of interest captured from, for example the cell are not on the same polynucleotide strand but both have the same barcoding sequence encoded in each strand, so the relationship between the sequences is captured.

In one embodiment the cDNA is synthesised employing an enzyme, for example a reverse transcriptase.

In one aspect, the present disclosure provides a method of covalently capturing a first and/or second polynucleotide sequence comprising the steps of:
b) annealing a first and second polynucleotide sequence (such as an RNA sequence) from, for example a cell, to its complementary sequence in the single stranded polynucleotide probe portions of a duplex probe molecule of the present disclosure as described above; and
c) employing a reverse transcriptase to synthesise a complementary strand to each annealed polynucleotide by extending from the single stranded polynucleotide portions of the duplex molecule in the 3′ direction along the length of the annealed polynucleotide sequence.

In one embodiment, the single stranded portions of the duplex molecule are extended until the leader sequence(s) of the annealed polynucleotide sequences are synthesised. Advantageously, the leader sequences provide a convenient sequence for which suitable sequencing primers are available or can be designed.

In one embodiment the method comprises the further step of preparing a fully double stranded molecule by synthesising the DNA sequence corresponding to the cDNA captured in strand-one and strand-two, for example employing a polymerase, such that both the sense and antisense strand of the “duplex probe molecule” are double stranded along their whole length and each strand encodes both polynucleotide captured from the cell.

Fully double stranded as employed herein refers to a molecule comprising two complementary polynucleotide strands which bind through base pairing to form a double stranded molecule wherein each strand is approximately or substantially the same length.

Thus in a further embodiment the method comprises an additional step of synthesising the corresponding double stranded DNA to said first and second cDNA sequences captured from the cell, for example employing a polymerase, such as DNA polymerase, in particular Sulfolobus polymerase, Mako polymerase, Taq polymerase, Klenow exo neg DNA polymerase or the like. This provides a fully double stranded molecule with each strand comprising the first and second polynucleotide captured from the cell linked by a barcode i.e. the captured polynucleotides are physically linked.

In one embodiment, the first and/or second polynucleotide sequences captured contain one or more coding regions, for example that encode antibodies, antibody fragments or antibody regions, such as antibody variable domains.

In one embodiment, the first and/or second polynucleotide sequences captured from the cell encode an antibody VH region and an antibody VL region, for example the first captured polynucleotide encodes a VH and the second captured polynucleotide encodes a VL. Advantageously, in the final double stranded molecule the linking of two variable regions via a unique barcode allows the antibody sequences to be physically coupled together as a discrete unit.

A further advantage of the claimed method is its ability to capture polynucleotide sequences with unknown sequence portions such as the variable regions of an antibody, based on the complementary binding of the duplex molecules to the known portions of the polynucleotide sequences, such as constant regions. This enables the duplex molecule to be used as a bait to selectively ‘fish’ for polynucleotide sequences having certain desirable sequences in a sample, such as a target cell.

At any stage after the first and second polynucleotides from the cell have been captured then the polynucleotide strands can be sequenced, for example employing next generation sequencing techniques, to recover the genetic information therein. In one embodiment the sequencing is performed after the cDNA complementing the captured polynucleotide has been synthesised employing reverse transcriptase, in particular before the complementary double strand to said cDNA is synthesised (i.e. where one variable region is encoded in each strand and both strands have the same barcode).

In one embodiment both variable regions are encoded in the same strand of polynucleotide (DNA) when the sequencing is performed. However, generally sequencing will be formed after a cloning step.

Before or after sequencing the polynucleotides may be amplified, for example employing a technique such as PCR.

Additionally, or alternatively the method may further comprises a cloning step, wherein the first and/or second polynucleotide(s) captured from the cell is/are cloned and expressed, for example in a host cell or from a transcriptionally active polynucleotide. That-is-to-say, one or both of the separate strands of the duplex probe molecule are expressed.

In one embodiment the present disclosure relates to a molecule obtained or obtainable from a method described herein.

Features of the duplex probe molecule described in the context of the method of the present disclosure apply where appropriate to the molecule per se and vice versa.

The method may be used to simultaneously capture from a cell a pair of heavy and light chain mRNA polynucleotide from an antibody producing cell, which together form a functional antibody binding fragment. After the annealing step, the duplex molecule with the annealed polynucleotide sequences can then be retrieved, and the reverse transcription step performed in vitro.

In one embodiment the method or part thereof is performed in an individual container, for example a cell, a droplet or a well comprising a duplex probe molecule of the present disclosure.

In one embodiment a cell employed is a B cell (including a lysed B cell).

In another aspect, there is provided a kit comprising one or more duplex probe molecules as described above and optionally reagents and/or instructions for use.

### Definitions

“Synthesise the complementary polynucleotide sequence from the relevant primer along the length of strand-one” as used herein (in particular in step e)) refers to the act of generating the complementary sequence by extending from the primer annealed to strand-one to form a section of double strand sequence which encodes in each strand the relevant genetic information, such as the barcode and the restriction site, where relevant. The extension step may be performed, for example by employing a nucleic acid polymerase, which when supplied with a sufficient quantity of nucleotides, utilises the relevant section of strand-one as a template sequence and synthesises the complementary sequence thereto. An analogous definition may also apply to strand-two, depending on the method employed.

In the context of the present disclosure, the complementary polynucleotide sequence is typically synthesised along part or all of the length of the relevant template, for example in step e) a complementary sequence is synthesised to part of strand-one, such as the part comprising the barcoding sequence and the restriction site to form a section which is double-stranded at the 5′end of the molecule.

“Complementary sequence” refers to a sequence which is capable of hybridising to a sequence which it complements, for example under stringent conditions, in particular, it includes polynucleotide sequences which contain the same genetic information and can hybridise to each other, such as cDNA and mRNA. In one embodiment it refers to the sense strand and antisense strand relationship between DNA molecules or between RNA molecules.

The terms “restriction site”, “restriction enzyme site” or “restriction enzyme recognition site” are used interchangeably in the specification and refer to a sequence motif which is the substrate for a restriction enzyme. Upon recognition of the relevant sequence motif, the restriction enzyme then cuts or cleaves the polynucleotide strand at a fixed position within the sequence motif. The resultant cut end of a polynucleotide strand can be either sticky or blunt depending on the restriction enzyme.

“Sticky end” as used herein refers to cut end with a protruding 5′ or 3′ overhang, whereas a “blunt end” refers to a cut end with no overhang. Restriction sites that produce sticky ends, in particular non-palindromic sticky ends are advantageous because molecules with compatible sticky ends can be ligated together more efficiently than blunt ends. Non-palindromic sticky ends, in particular may result in fewer “mismatched” ligations occurring as during the ligating step and helps to ensure that strands one and two are ligated in the correct orientation with respect to each other.

Examples of non-palindromic restriction sites and their associated restriction enzymes are shown in Table 1 below.

TABLE 1
Restriction Enzymes with non-palindromic recognition sites.

| Enzyme | Recognition Sequence (5′ to 3′) | Enzyme | Recognition Sequence (5′ to 3′) |
|---|---|---|---|
| AciI* | CCGC(−3/−1) | BtsCI* | GGATG(2/0) |
| AcuI* | CTGAAG(16/14) | BtsI* | GCAGTG(2/0) |
| AlwI* | GGATC(4/5) | BtsIMutI* | CAGTG(2/0) |
| BaeI* | (10/15)ACNNNNGTAYC(12/7) | CspCI* | (11/13)CAA GTGG(12/10) |
| | SEQ ID NO: 7 | | SEQ ID NO: 8 |
| BbsI* | GAAGAC(2/6) | Earl* | CTCTTC(1/4) |
| BbvCI* | CCTCAGC(−5/−2) | EciI* | GGCGGA(11/9) |
| BbvI* | GCAGC(8/12) | EcoP15I* | CAGCAG(25/27) |
| BccI* | CCATC(4/5) | FauI* | CCCGC(4/6) |
| BceAI* | ACGGC(12/14) | FokI* | GGATG(9/13) |
| BcgI* | (10/12)CGANNNNNNTGC(12/10) | FspEI* | CC(12/16) |
| | SEQ ID NO: 9 | | |
| BciVI* | GTATCC(6/5) | HgaI* | GACGC(5/10) |
| BcoDI* | GTCTC(1/5) | HphI* | GGTGA(8/7) |
| BfuAI* | ACCTGC(4/8) | HpyAV* | CCTTC(6/5) |
| BmgBI | CACGTC(−3/−3) | I-CeuI* | TAACTATAACGGTCCTAAGGTAG |
| | | | CGAA(−9/−13) SEQ ID NO: 10 |
| BmrI* | ACTGGG(5/4) | I-SceI* | TAGGGATAACAGGGTAAT(−9/−13) |
| | | | SEQ ID NO: 11 |
| BpmI* | CTGGAG(16/14) | LpnPI* | CCDG(10/14) |
| Bpul0I* | CCTNAGC(−5/−2) | MboII* | GAAGA(8/7) |
| BpuEI** | CTTGAG(16/14) | MlyI | GAGTC(5/5) |
| BsaI(1)* | GGTCTC(1/5) | MmeI* | TCCRAC(20/18) |
| BsaI- | GGTCTC(1/5) | MnlI* | CCTC(7/6) |
| HF®* | | | |
| BsaXI* | (9/12)ACNNNNNCTCC(10/7) | MspJI* | CNNR(9/13) |
| | SEQ ID NO: 12 | | |
| BseRI* | GAGGAG(10/8) | Nb.BbvCI* | CCTCAGC |
| BseYI* | CCCAGC(−5/−1) | Nb.BsmI* | GAATGC |
| BsgI* | GTGCAG(16/14) | Nb.BsrDI* | GCAATG |
| BsmAI* | GTCTC(1/5) | Nb.BtsI* | GCAGTG |
| BsmBI* | CGTCTC(1/5) | NmeAIII* | GCCGAG(21/19) |
| BsmFI* | GGGAC(10/14) | Nt.AlwI* | GGATC(4/−5) |
| BsmI* | GAATGC(1/−1) | Nt.BbvCI* | CCTCAGC(−5/−7) |
| BspCNI* | CTCAG(9/7) | Nt.BsmAI* | GTCTC(1/−5) |
| BspMI* | ACCTGC(4/8) | Nt.BspQI* | GCTCTTC(1/−7) |
| BspQI* | GCTCTTC(1/4) | Nt.BstNBI* | GAGTC(4/−5) |
| BsrBI | CCGCTC(−3/−3) | Nt.CviPII* | (0/−1)CCD |
| BsrDI* | GCAATG(2/0) | PI-PspI* | TGGCAAACAGCTATTATGGGTAT |
| | | | TATGGGT(−13/−17) |
| | | | SEQ ID NO: 13 |
| BsrI* | ACTGG(1/−1) | PI-SceI* | ATCTATGTCGGGTGCGGAGAAA |
| | | | GAGGTAAT(−15/−19) |
| | | | SEQ ID NO: 14 |
| BssSαI* | CACGAG | PleI* | GAGTC(4/5) |
| BssSI* | CACGAG(−5/−1) | SapI* | GCTCTTC(1/4) |
| BtgZI* | GCGATG(10/14) | SfaNI* | GCATC(5/9) |
| BtsαI* | GCAGTG | | |

Numbers in parentheses indicate the point of cleavage, for example GGTCTC(1/5) indicates cleavage at: 5′ . . . GGTCTCN/ . . . 3′; and 3′ . . . CCAGAGNNNN/ . . . 5′ SEQ ID NO: 29.

Restriction sites which produce sticky ends when cut with their respective restriction enzymes are marked with an asterisk in Table 1. These restriction sites are particularly suitable for use in the method and molecules of the present disclosure.

The terms “non-palindromic site”, “non-palindromic restriction site” or “non-palindromic restriction enzyme site” as used herein refer to the recognition site of a restriction enzyme that recognizes a non-palindromic sequence and cuts the sequence to provide two ends which are non-palindromic The same restriction enzyme may be used to cut strand-one and strand-two, or a different enzyme may be used to cut each strand, provided that the sticky ends generated in both strands are compatible with each other and allow strands one and two to be ligated together. However, it may more convenient and simpler to use the same restriction enzyme to cut both strands. Accordingly, in one embodiment the same restriction enzyme is used to cut both strand one and strand two.

Clearly the restriction enzyme employed needs to correspond with or be suitable to the restriction site encoded.

Additional base pairs upstream and/or downstream of the restriction sites may be included in the relevant strand after the site to facilitate the restriction enzymes to cut their restriction sites efficiently.

The number of additional base pairs may be one, two, three, four, five, six or more depending on the restriction enzyme used. In one embodiment strand-one before being cut with a restriction enzyme comprises junk DNA between the restriction site and the 5′ end of the sequence. In one embodiment strand-two before being cut with a restriction enzyme comprises junk DNA between the restriction site and the 5′ end of the sequence. Junk DNA as employed herein refers to non-coding DNA, for example 6 to 12 base pairs in length.

Thus, in one embodiment the at least 6 base pairs downstream of the restriction site are provided and/or 6 base pairs upstream of the restriction site (i.e. moving in a direction away from the restriction site and barcode) is junk DNA.

The term “DNA ligase,” as used herein, refers to a family of enzymes which catalyze the formation of the covalent phosphodiester bond between two distinct DNA strands, i.e. a ligation reaction. Two prokaryotic DNA ligases, namely the ATP-dependent T4 DNA ligase (isolated from the T4 phage), and the NAD⁺-dependent DNA ligase from E. coli, have become indispensable tools in molecular biology applications and are suitable for use in a ligase reaction in the method of the present disclosure. Both enzymes catalyse the synthesis of a phosphodiester bond between the 3′-hydroxyl group of one polynucleic acid, and the 5′-phosphoryl group, of a second polynucleic acid. T4 DNA ligase is commercially available from at least USB and New England Biolabs.

The terms “covalently capturing a first and/or second polynucleotide sequence” or “covalent capture of a first and/or second polynucleotide sequence” as used herein refers to a process wherein the complementary sequence of the first and/or second polynucleotide sequence is synthesised and covalently attached to a duplex molecule of the present disclosure, in particular by extending the polynucleotide probe or probes therein. Since the complementary sequence is physically joined to the duplex molecule, it is said to be “hard coded” as part of the duplex molecule itself. In some instances the two captured polynucleotide sequences are on separate polynucleotide strands of the duplex molecule. Where the duplex molecule is completely or fully double stranded it hard codes each captured polynucleotide into both the sense and antisense strand of the molecule.

Reverse transcriptase as employed herein refers to an enzyme employed to generate cDNA from an RNA template. Reverse transcriptases useful in the present disclosure include, but are not limited to, reverse transcriptases from HIV, HTLV-1, HTLV-II, FeLV, FIV, SIV, AMV, MMTV, MoMuLV and other retroviruses (see Levin (1997) Cell, 88:5-8; Verma (1977) Biochim. Biophys. Acta, 473:1-38; Wu et al. (1975) CRC Crit. Rev. Biochem., 3:289-347).

The term “polynucleotide” as employed herein refers to a biopolymer of covalently bonded nucleic acids monomers, for example 3 or more monomers including RNA polynucleotides such as messenger RNA (mRNA) and DNA polynucleotides, such as “linear” DNA polynucleotides and plasmid DNA (pDNA).

In some embodiments, the methods of the present disclosure involve performing polynucleotide sequencing steps in order to identify unknown polynucleotide sequences or unknown portions of the primers, a polymerase, and a reverse transcriptase.

The term “about,” as used herein, generally refers to a range that is 15% greater than or less than a stated numerical value within the context of the particular usage. For example, “about 10” would include a range from 8.5 to 11.5. In one embodiment about represents +/−10%.

“Comprising” in the context of the present specification is intended to mean “including”. Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present application claims priority from GBXXX filed 3 Jul. 2018, the contents of which is incorporated by reference. The priority application may used as basis for corrections of mistakes in the present specification.

The invention will now be described with reference to the following examples, which are merely illustrative and should not in any way be construed as limiting the scope of the present invention.

### DESCRIPTION OF THE FIGURES

FIG. 1 is a diagrammatic representation of duplex probe molecule

FIG. 2 is a diagrammatic representation of the steps involved in the synthesis of a duplex probe molecule of FIG. 1

FIG. 3A is a diagrammatic representation of the synthesis of a duplex probe molecule of the present disclosure

FIG. 3B is a diagrammatic representation of the synthesis of a duplex probe

FIG. 4A is a diagrammatic representation of the duplex probe molecule of the present disclosure without a barcode

FIG. 4B is a diagrammatic representation of a duplex probe molecule according to the present disclosure with flanking barcodes.

FIG. 4C is a diagrammatic representation of a duplex probe molecule according to the present disclosure with a central barcode

FIG. 5A is a diagrammatic representation of a duplex probe molecule (no barcode) with polynucleotides.

FIG. 5B is a diagrammatic representation of a duplex probe molecule (flanking barcode(s)) with polynucleotides.

FIG. 5C is a diagrammatic representation of a duplex probe molecule (barcode in the central core) with polynucleotides.

FIG. 6A is a diagrammatic representation of extending a probe portion of a duplex probe molecule using captured RNA as the template, without a barcode.

FIG. 6B is a diagrammatic representation of extending a probe portion of a duplex probe molecule using captured RNA as the template, with flanking barcode(s).

FIG. 6C is a diagrammatic representation of extending a probe portion of a duplex probe molecule using captured RNA as the template, barcode in the central core.

FIG. 7 is a summary and comparison of the process of capturing DNA employing three different types of duplex probe molecules.

FIG. 8A is a diagrammatic representation of a duplex probe molecule (no barcode) with polynucleotides captured after treatment with RNAse, to remove the RNA.

FIG. 8B is a diagrammatic representation of a duplex probe molecule (flanking barcode(s)) with polynucleotides captured after treatment with RNAse, to remove the RNA.

FIG. 8C is a diagrammatic representation of a duplex probe molecule (barcode in the central core) with polynucleotides captured after treatment with RNAse, to remove the RNA.

FIG. 9 shows strands in molecules of FIG. 8 are separated using heating.

FIG. 10 shows, alternatively, the molecule can be rendered fully double stranded employing a polymerase.

FIG. 11 is a series of photographs showing the results of gel electrophoresis experiments performed to check that the oligos obtained after each step of the method of the present disclosure correspond to their expected sizes. (A) gel photograph taken after annealing step. (B) gel photograph taken after the extension step.

FIG. 12A is a photograph showing the results of a gel electrophoresis experiment performed to check that the oligos obtained for CH1 and CK samples match their expected sizes. A 5% agarose gel was used. For the annealing step, 6 μl of long primer and 6 μl of short primer together with 30 μl dH₂O was used for each sample. The sample was incubated on ice to allow the short primers to anneal to the long primers. An aliquot of 7 μl was kept aside to be run on an agarose gel.

For the extension step, 5 μl of buffer, 5 μl of dNTPs and 5 μl of Klenow polymerase was added to the remaining 35 μl of sample. The mixture was then incubated at 25° C. for 1 hour.

FIG. 12B is a schematic diagram showing the desired products from each step of the method and their expected sizes.

FIG. 13 shows a series of photographs showing the results of gel electrophoresis experiments using a different set of primers from those used in FIG. 12 to check that the oligos obtained for CH1 and CK samples match their expected sizes. A 5% agarose gel was used.

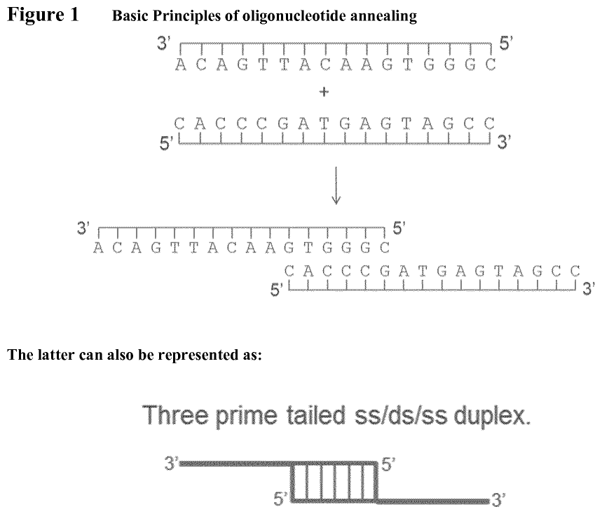

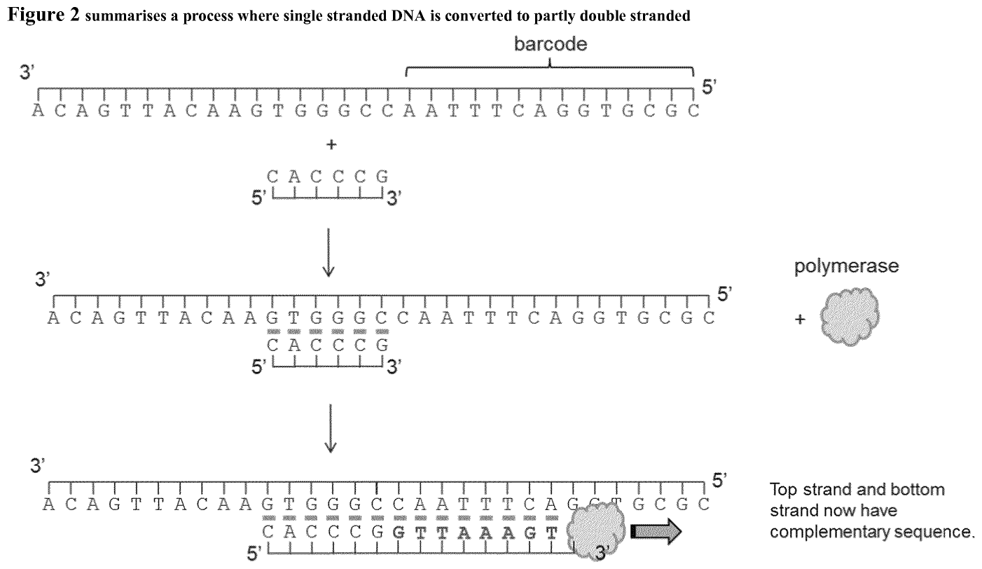

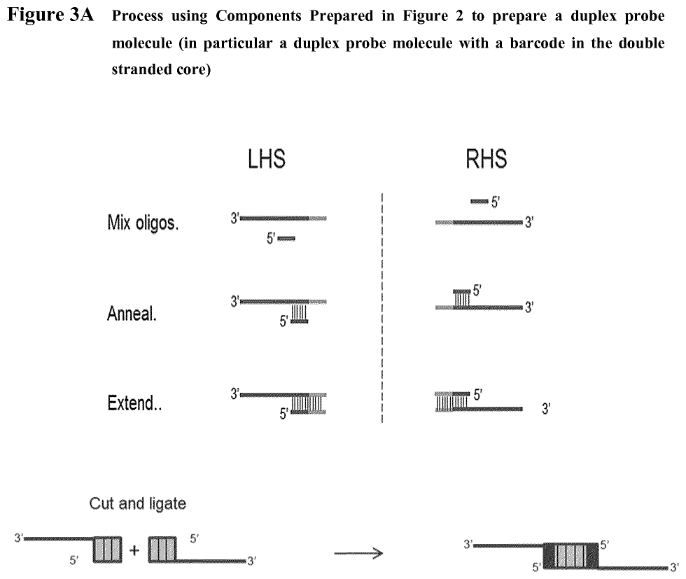

The same amount of each reaction component as before was used. However, a shorter annealing program was used this time.

Sample 1: 5 μl run directly after extension step

Sample 2: 10 μl put through minelute 1× column, followed by elution in 15 μl. All samples run without isopropanol

Sample 3: 10 μl put through put through minelute 1× column, followed by elution in μl. All samples run with isopropanol

About 500 ng of sample was recovered from 10 μl. Based on the results, it appeared that clean up without isopropanol produced similar results to with isopropanol.

Hence, further clean up steps were performed without isopropanol.

FIG. 14A is a plasmid restriction map of EcoRI cut (linearised) pNAFH.

Expected size of fragment cut with EcoRI and BbvCI is 852 bp.

FIG. 14B is a gel electrophoresis photograph showing the results of restriction digests of pNAFH using EcoRI and BbvCI performed at a series of different temperatures ranging from 30 to 37° C.

FIG. 15 shows a gel electrophoresis photograph of the final duplex molecule obtained using the new annealing temperature.

FIG. 16 shows capturing mRNA using oligo-dT (50 μM) or using the constant region-specific primer—heavy or kappa long primer (2 μM). Synthesised cDNA was used in a standard PCR reaction as using primers detailed in Example 2.

FIG. 17 shows annealing and extension of long and complementary short primers with DNA polymerase

FIG. 18 shows upper band in POC ligation is double the size of lower band. Predicted incomplete ligation of heavy and light chain sides. Ligated size ˜125 bp. Non-ligated ˜75 bp. The control shows product pre-ligation

FIG. 19 shows PCR products after 2 rounds of PCR amplification using primer conditions disclosed herein

FIG. 20 shows data exemplifying the ability to capture both heavy and light chain mRNA using the duplex probe molecule of the present disclosure. The band observed at 1000 bp is the correct molecular weight for a captured VH and VL. This band was generated using the forward primers that are complementary to the 5′-region upstream of either VH or VL (kappa) in a rabbit. Arrows indicate the position of dominant banding on the gel at positions consistent with a duplex capturing template via one (500 bp) or both (1,000 bp) polynucleotide tails.

### EXAMPLES

### Example 1 First Stage of Process for Preparation of Duplex Molecule According to Method of Present Disclosure

The duplex molecule was constructed using a ‘long’ primer containing either heavy or light chain constant region specific sequence, an oligo annealing sequence, a barcoded (or fixed sequence in the roof of concept conditions and restriction site BbvCI.

To be carried out with constant heavy-specific primers and constant-kappa specific primers.

Step 1: Anneal short primer to long primer. Step 2. Used polymerase to extend double-stranded DNA.

Step 3: Digest DNA for subsequent ligation with other-handed side of duplex molecule.

The following 4 oligonucleotides were synthesised by Sigma Aldrich. All sequences are shown in the 5′ to 3′ direction.

21889 CH-BC long SEQ ID NO: 1:
CATATCGCCTCAGCNNNNNNNNNNNNNNCTGCACACGGGCGCGTCCCGTG
GGAAGACTGACGGACGCCTTAGGTTG
21888 CK-BC long SEQ ID NO: 2:
AGCTATAGCTGAGGNNNNNNNNNNNNNNGTCCGAGCTGCGCCCTGCGGGA
AGATGAGGACAGTAGGTGCAACTGG
21887 CH-BC short SEQ ID NO: 3
GGGACGCGCCCGTGTGCAG
21886 CK-BC short SEQ ID NO: 4
CGCAGGGCGCAGCTCGGAC

The bases in italics (i.e. not underlined) represent junk sequence which was added to enhance the efficiency of the restriction digests. The underlined bases represent the BbvCI restriction site. The NNNN region in the long oligos is the barcoding region, wherein each N represents any one of bases A, G, T or C. The bold text represents the priming sequence. The underlined and italic bases represent the polynucleotide probe sequences.

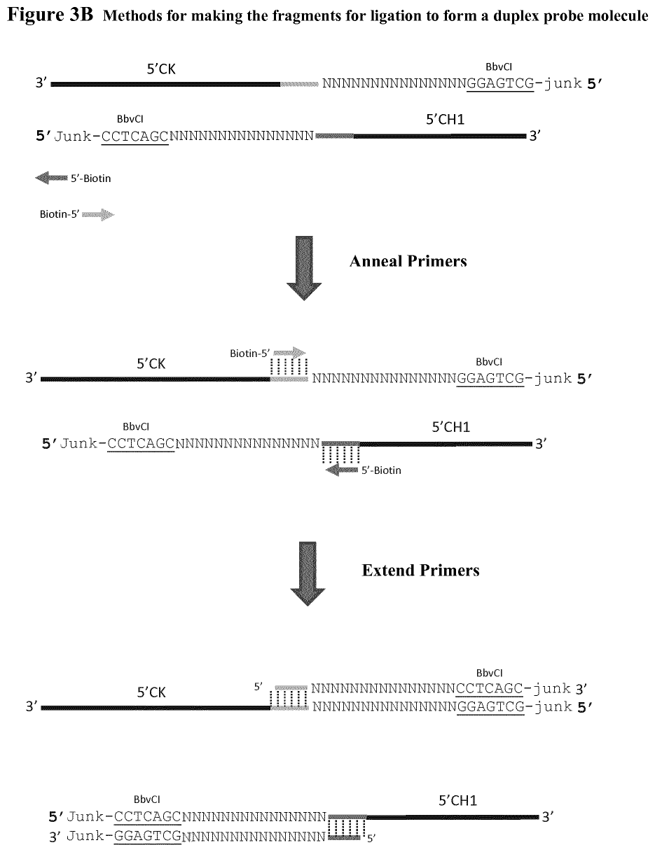

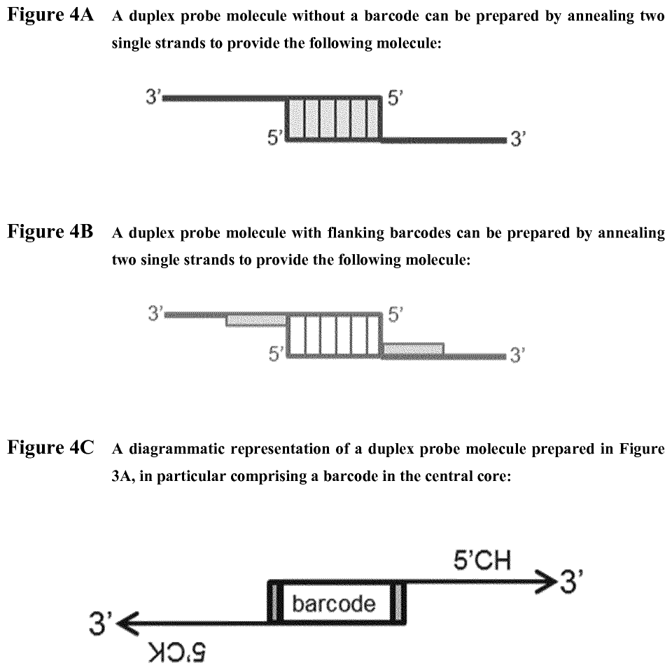

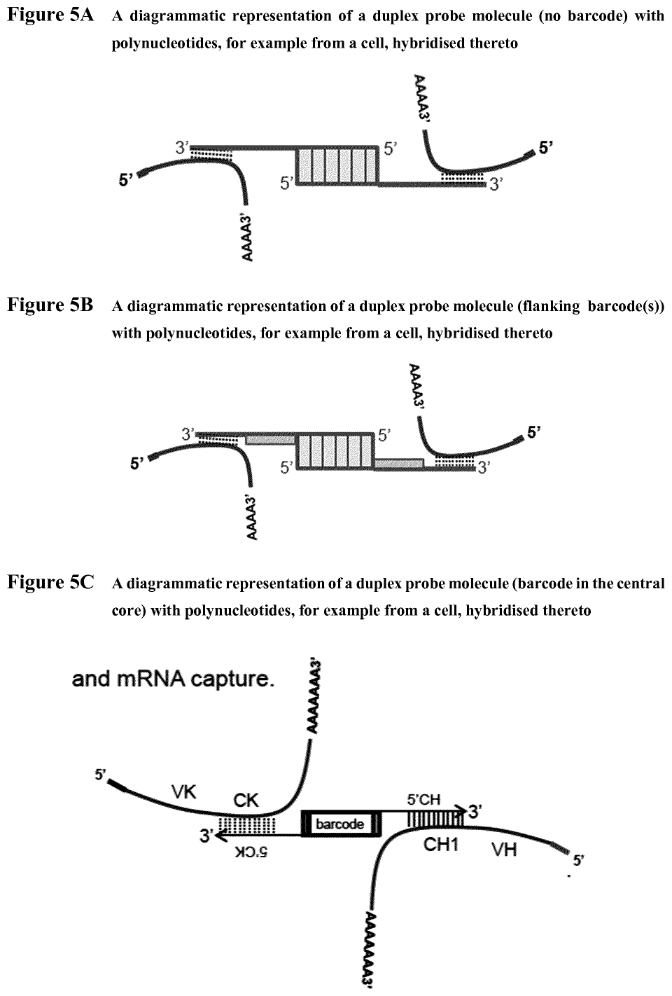

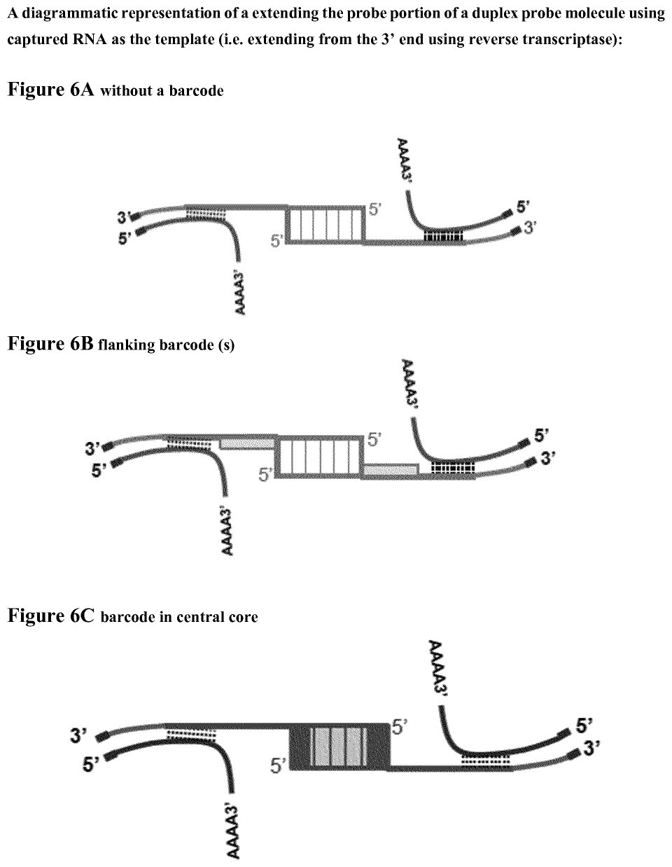

The first stage of the process for preparing the duplex molecule is shown in FIG. 3B.

First, 21889 CH-BC long SEQ ID NO: 1 mixed with 21887 CH-BC short SEQ ID NO: 3, whilst 21888 CK-BC long SEQ ID NO: 2 was separately mixed with 21886 CK-BC short SEQ ID NO: 4. The 2 mixtures were heated to 98° C. and then left to slowly cool in order to allow the short oligos (primers) to anneal to the long oligos.

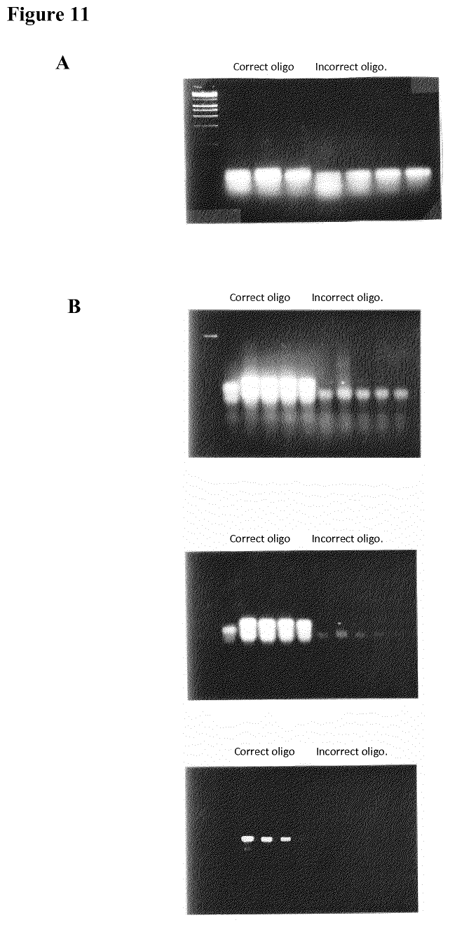

Klenow fragment exo negative (New England Biolabs) was then used to extend the 3′ ends of the short oligos. The reactions were done separately for each long:short oligo mixture. This generates the two halves of the final duplex molecule. At this stage of the process, the NNNN barcode region is hard coded to both top and bottom strands of the duplex, whilst the long oligo 3′ end remains single stranded. To check that the size of the products matched their expected sizes, aliquots of the reaction mixtures were taken and agarose gel electrophoresis was carried out in order. The results of the agarose gel electrophoresis experiments are shown in FIG. 11. As can be seen, the smaller products are the incorrect oligos which represent long oligos that do not have the annealed and fully extended short oligos. The correct oligos represent the long oligos with the properly extended short oligos annealed.

### Example 2 Constructions of Duplex Probe Molecules without Barcodes

Further primers employed to construct the duplex molecule, which did not contain a barcode:

SEQ ID NO: 15
AGCTATAGCTGAGGATGCAGTGCTGCCAGTCCGAGCTGCGCCCTGCGGGA
AGATGAGGACAGTAGGTGCAACTGG
SEQ ID NO: 16
ATATCGCCTCAGCCGATGCCTGTAGCCTGCACACGGGCGCGTCCCGTGGG
AAGACTGATGGAGCCTTAGGTTGCC

The long primers are based on the IMGT Rabbit constant region sequences.

Taken from: http://www.imgt.org/IMGTrepertoire/Proteins/index.php#D

```
SEQ ID NO: 17
                  R D P V A P S V L L F P P S K E
IGKC2*01 cgt gat cca gtt gcg cct tct gtc ctc ctc ttc cca cca tct aag gag
                  X D P V A P T V L I F P P A A D
IGKC1*01 ngt gat cca gtt gca cct act gtc ctc atc ttc cca cca gct gct gat
                    G Q P K A P S V F P L A P C C G
IGHG*01 FCH1 ggg caa cct aag gct ccg tca gtc ttc cca ctg gcc ccc tgc tgc ggg
RDPVAPSVLLFPPSKEXDPVAPTVLIFPPAADGQPKAPSVFPLAPCCG
SEQ ID NO: 18
cgtgatccagttgcgccttctgtcctcctcttcccaccatctaaggagngtgatccagttgcacctactgtcctca
tcttcccaccagctgctgatgggcaacctaaggctccgtcagtcttcccactggccccctgctgcggg
```

The following is a description of the method for constructing a non-barcoded duplex molecule. The heavy and light chain sections were constructed separately and subsequently ligated together to form the full duplex.
1. Denature primers at 95° C. for 5 minutes

Reaction Mixture:

Proof of Concept (POC)

| Heavy (H) | Kappa (K) |
|---|---|
| 0.5 μl of 100 μM CH-POC-long3 primer | 0.5 μl of 100 μM CK-POC long primer |
| (Table 1) | (Table 1) |
| 0.5 μl of 100 μM CH-BC short primer | 0.5 μl of 100 μM CK-BC short primer |
| (Table 1) | (Table 1) |
| 24 μl sterilised distilled water | 24 μl sterilised distilled water |

Control

| Heavy (HC) | Kappa (KC) |
|---|---|
| 0.5 μl of 100 μM CH-POC-1ong3 primer | 0.5 μl of 100 μM CK-POC long primer |
| (Table 1) | (Table 1) |
| 0.5 μl of 100 μM CK-BC short primer | 0.5 μl of 100 μM CH-BC short primer |
| (Table 1) | (Table 1) |
| 24 μl sterilised distilled water | 24 μl sterilised distilled water |

2. Heat activate 25 μl per reaction, of KOD Hot Start Master Mix (Merck, MA, USA) at 95° C. for 5 minutes.
3. Anneal long and short primers together at 70° C. for 2 minutes.
4. Add 25 μl of heat activated KOD per reaction. Run extension step at 70° C. for >10 minutes.
5. Run 2 μl of reaction on 2.3% agarose gel to observe intense band in extended conditions.
6. Column purify products using QIAGEN, PCR purification kit and protocol. Elute in 25 μl sterilised distilled water. See FIG. 17, which shows annealing and extension of long and complementary short primers with DNA polymerase. The control bands are dimmer, suggesting less or no extension of DNA due to non-complementary short primers that do not anneal to the longer primer.
7. Digest purified products with BbvCI (NEB) in 50 μl reaction for 20 hours at 37° C. according to manufacturer's instructions.
8. Column purify as before. Elute in 20 μl sterilised distilled water.
9. Ligate heavy and kappa parts together at a 1:1 ratio using T4 DNA Ligase (NEB) in a 50 μl reaction according to manufacturer's instructions. Incubate at 16° C. overnight.
10. Run out all product on agarose gel to observe formation of duplex ligation product. See FIG. 18 which shows upper band in POC ligation is double the size of lower band. Predicted incomplete ligation of heavy and light chain sides. Ligated size 125 bp. Non-ligated 75 bp. The control shows product pre-ligation.
11. Gel extract upper band exemplifying duplex molecule using QIAGEN Gel Extraction Kit and protocol: https://www.qiagen.com/gb/resources/resourcedetail?id=a72e2c07-7816-436f-b920-98a0ede5159a&lang=en

### Example 3 Incorporation of Clean Up Step

A second experiment was performed, whereby a clean-up step was incorporated after the extension step.

For the annealing step, 6 μl of long primer and 6 μl of short primer together with 30 μl dH₂O was used for each sample. The sample was incubated on ice to allow the short primers to anneal to the long primers. An aliquot of 7 μl was kept aside to be run on an agarose gel.

For the extension step, 5 μl of buffer, 5 μl of dNTPs and 5 μl of Klenow polymerase was added to the remaining 35 μl of sample. The sample was then incubated at 25° C. for 1 hour. Another aliquot of 7 μl was taken and kept for the agarose gel.

For the clean-up step, the samples were run through a Qiagen silica based column. A further aliquot of 7 μl was taken and kept for the agarose gel. The various aliquots were then run on a 5% agarose gel.

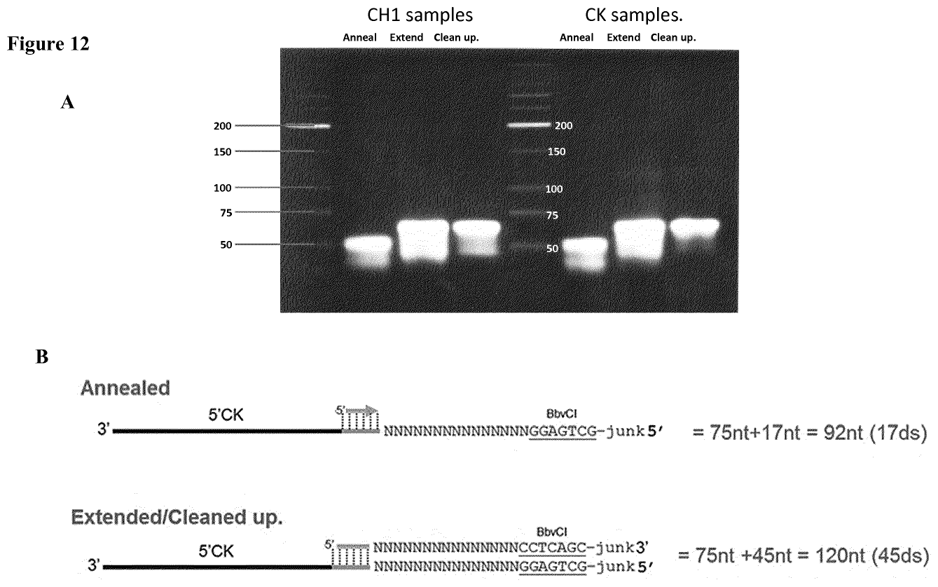

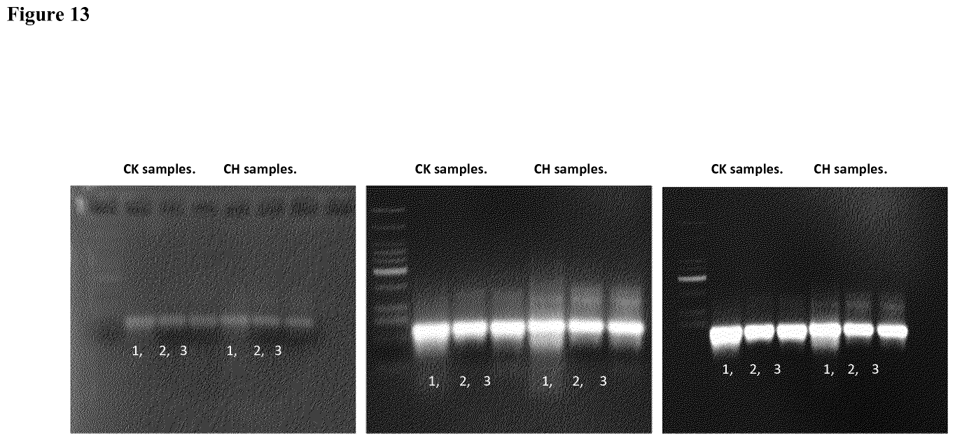

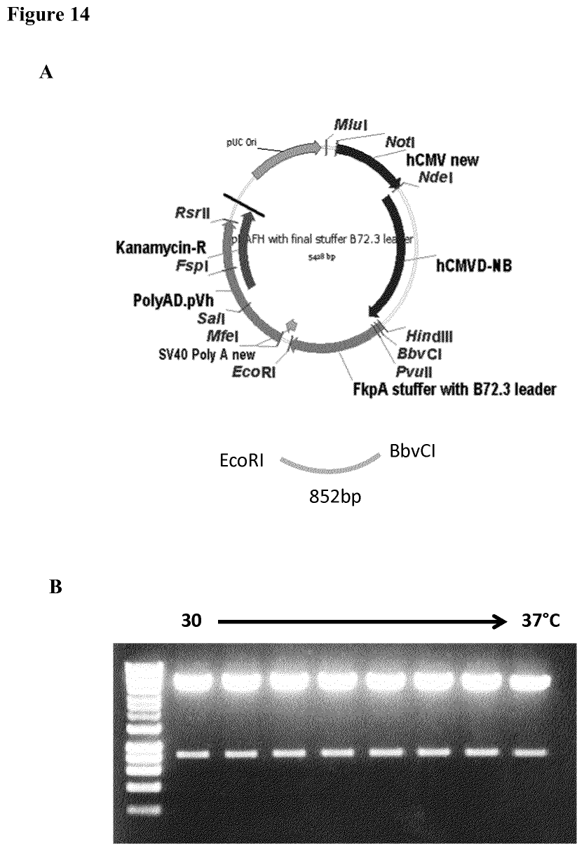

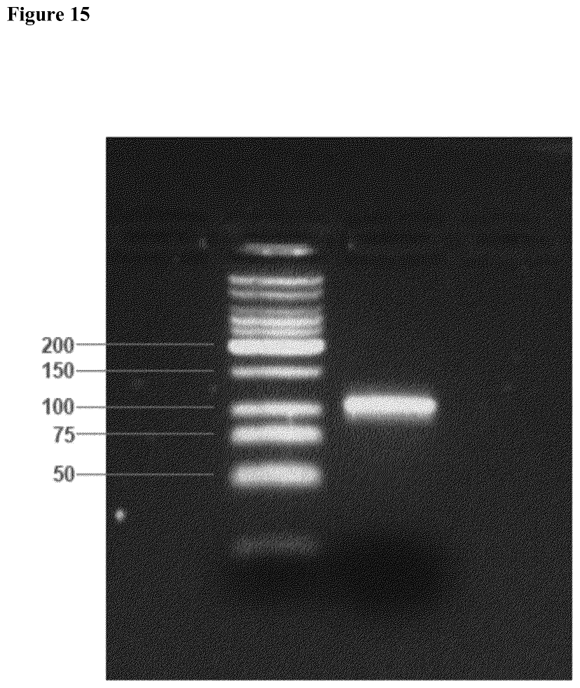

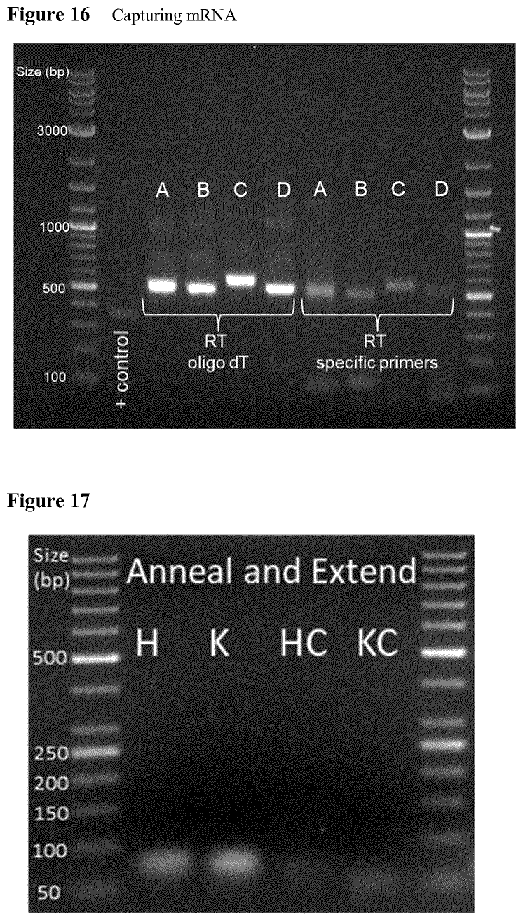

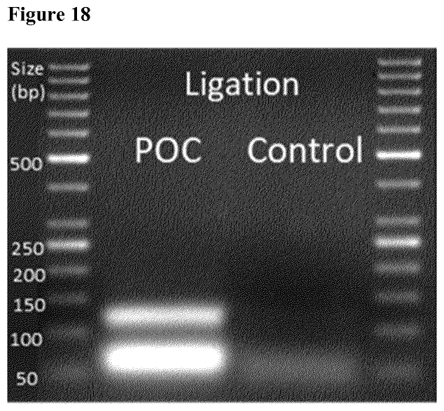

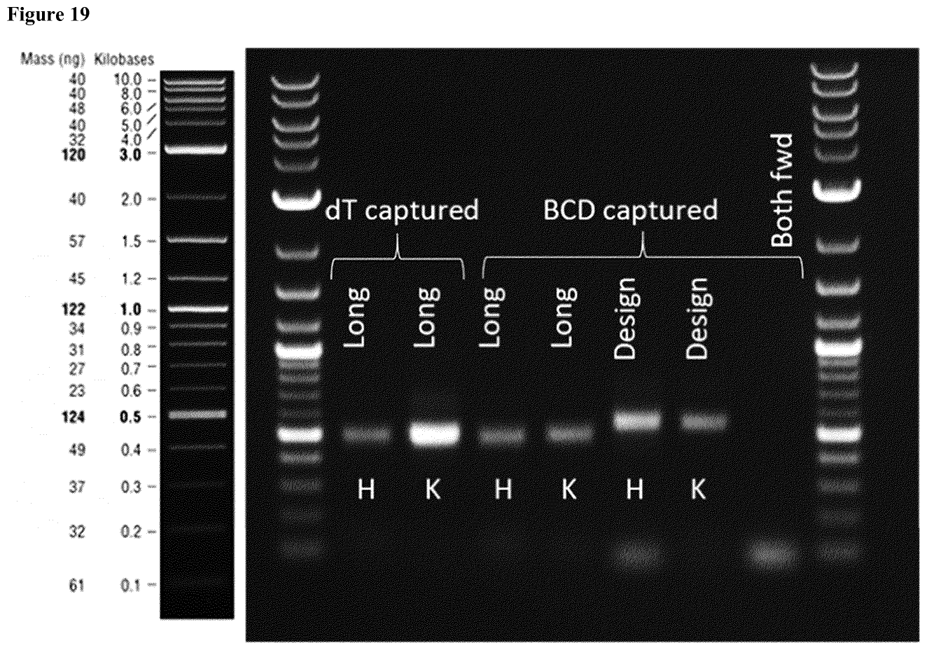

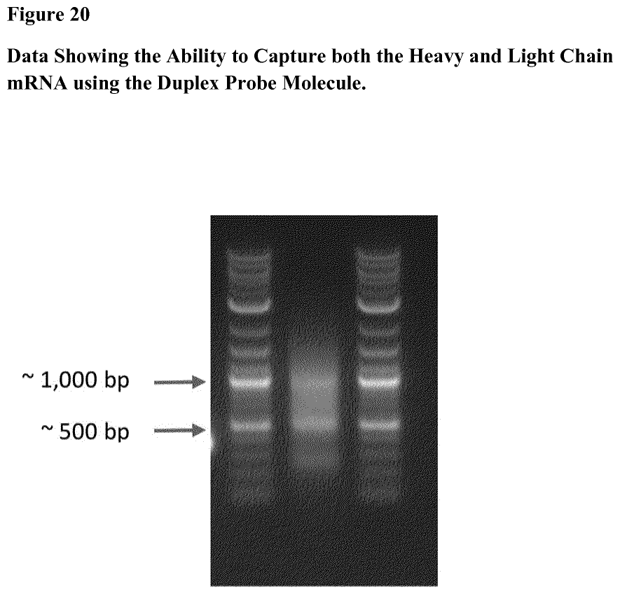

FIG. 12A shows the results of the agarose gel electrophoresis whilst FIG. 12B shows the expected sizes of the products after the annealing, extension and clean up steps.

As can be seen, the products match their expected sizes. The clean-up step also appears to have been effective given the reduction in the smearing of the band which suggests that the majority of the cleaned-up product is the desired long oligo with the annealed and fully extended short oligo.

Based on these results, a decision was made to incorporate a clean-up step in the process.

### Example 4—Further Optimisations to First Stage of Process

To further optimise the first stage of the process, a shorter annealing step and a modified clean up step using isopropanol were tested.

For the annealing step, 6 μl of long primer and 6 μl of short primer together with 30 μl dH₂O was used for each sample. The sample was incubated in a thermocycler and the temperature was reduced by 0.5° C. every 30 see to allow the short oligos to specifically anneal to the long oligos.

For the extension step, 5 μl of buffer, 5 μl of dNTPs and 5 μl of Klenow polymerase was added to the remaining 35 μl of sample. The sample was then incubated at 25° C. for 1 hour. An aliquot of 5 μl was taken and kept for the agarose gel.

For the clean-up step, 10 μl of each sample was put through a minelute 1× column and then eluted in 15 μl without isopropanol. Another 10 μl of each sample was also put through a minelute 1× column and then eluted in 15 μl with isopropanol.

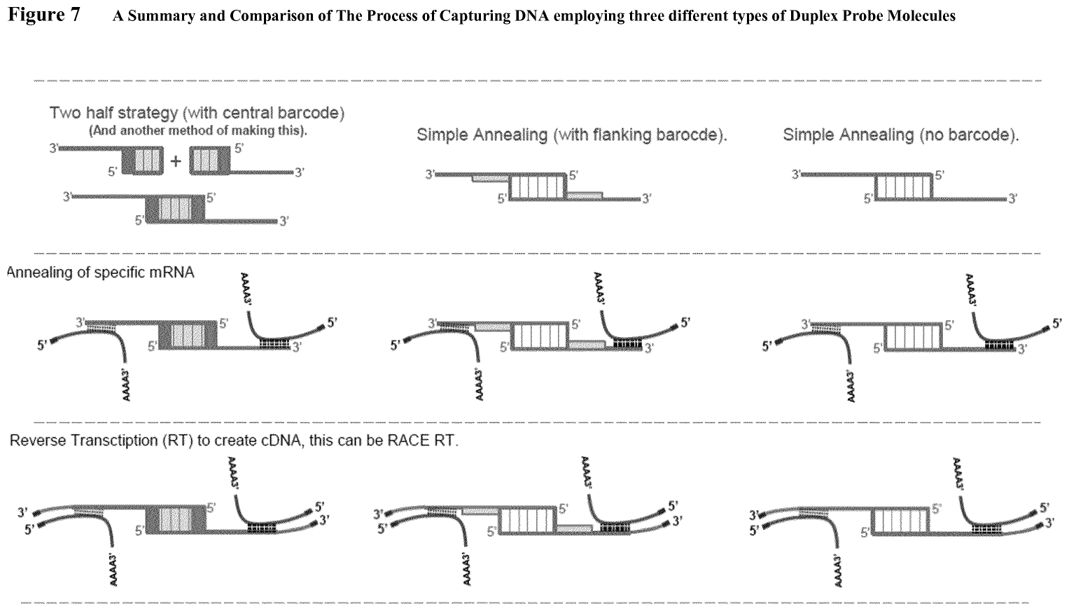

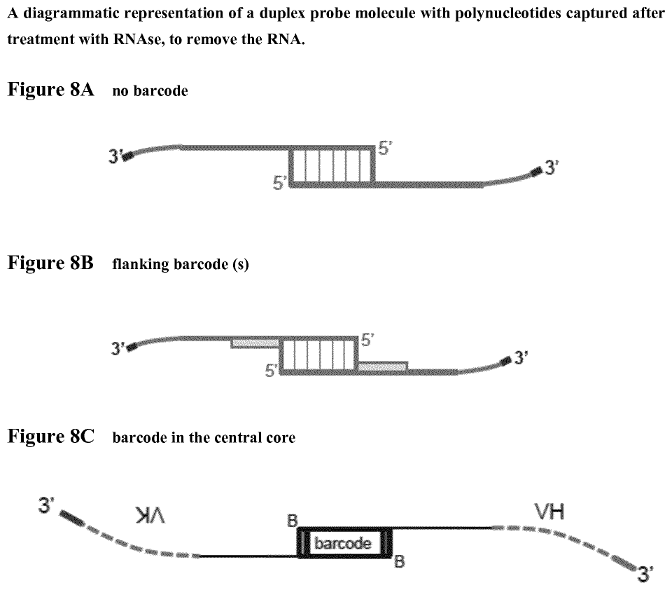

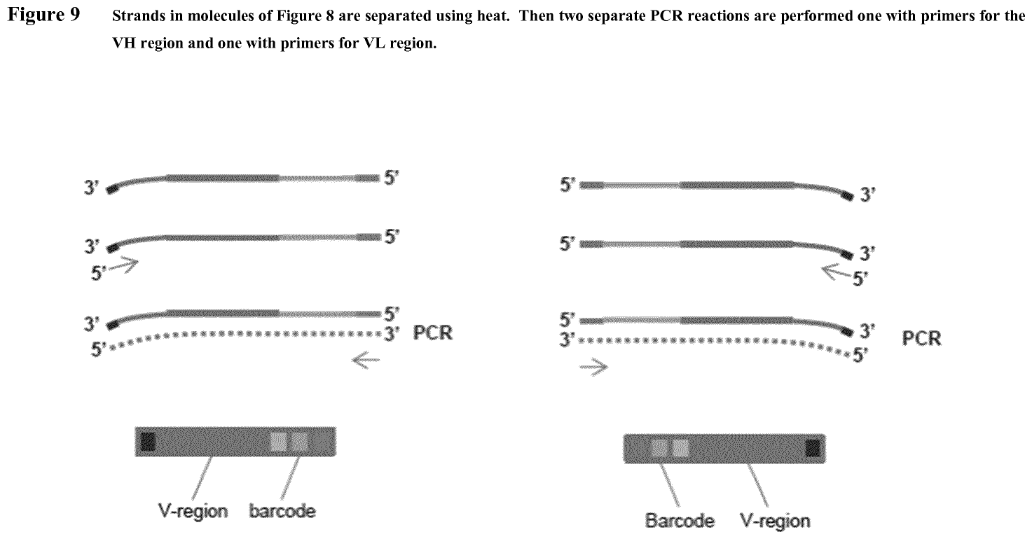

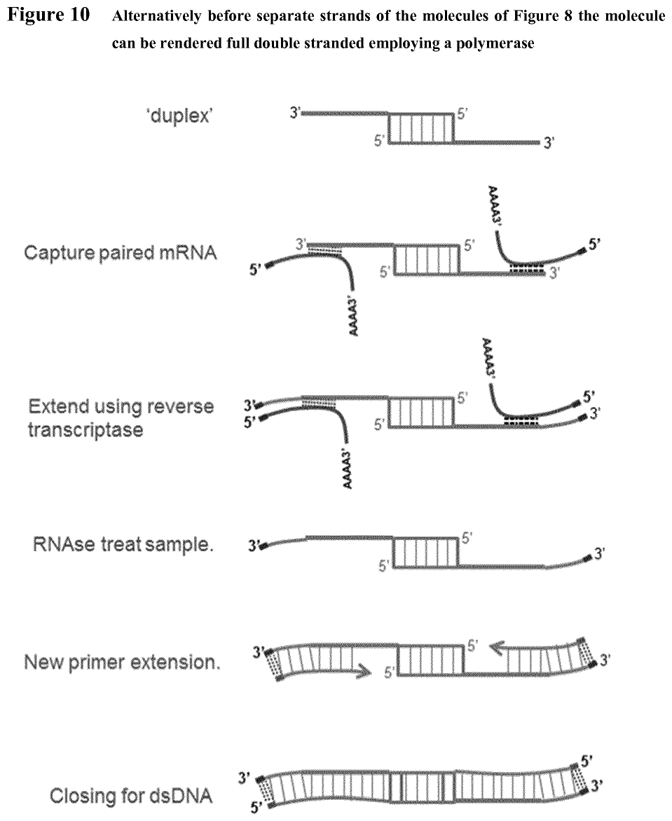

The 5 μl aliquots from the extension step and the entire 15 μl of cleaned up sample were then run on an agarose gel. The results of the gel electrophoresis are shown in FIG. 7.

As can be seen from the photographs, there is minimal difference between the cleaned up products that have been washed with isopropanol vs without isopropanol. Therefore, based on these results, future sample clean up steps were performed without isopropanol.

### Example 5 Determining Optimal Temperature for Performing Restriction Digests

To further optimise the digestion step, an experiment was performed to determine the optimum temperature for the restriction digest.

pNAFH was first digested with EcoRI to linearise the plasmid (see FIG. 15A). Next, separate aliquots of the linearised plasmid were incubated with BbvCI at a range of different temperatures ranging from 30° C. to 37° C. for 1 hour. The samples were then run on an agarose gel and the results of the electrophoresis experiment are shown in FIG. 15B.

As can be seen, there is minimal difference between the samples—in each case the 852 bp EcoRI/BbvCI fragment can be clearly seen and distinguished from the rest of the linearised plasmid.

Therefore, based on the results, it appears that the restriction digest can effectively be carried out at any temperature between 30 to 37° C., provided the digest is performed for at least an hour.

### Example 6 Capturing Polynucleotide Sequence in the Probe

Firstly, 4 oligo primers are obtained, two long and two short. The short ones are optionally biotinylated and designed to anneal in the middle of the longer oligos just upstream of the NNNN barcode region. These are performed as individual annealing reactions.

Both the long oligo primers contain recognition sites for the 5′ end of the first constant domains of antibody heavy and light chains, a uniquely designed primer site (green and yellow) followed by the 15 bp barcoded region which itself is followed by the BbvCI restriction enzyme site and some junk sequence for cutting off.

Klenow exo negative DNA polymerase is added to the long:short primer mixes and the short primers are extended to the ends of the long primers. As a result, the NNNN barcode region becomes “hard coded” to both top and bottom strand of the duplex, whilst the long primer 3′ ends remain still single stranded.

### Example 7 Use of a Duplex Probe Molecule

The duplex may be employed in a single cell captured into a small tube via micromanipulation or within a microfluidic droplet. Barcoding of cognate V-regions may be performed using the following steps:
1) capture of cognate pair mRNA via the duplex probe molecule, for example introduced into the cell or droplet;
2) extend the 3′ end of the probes in the duplex probe molecule using ‘Reverse Transcriptase’ enzyme, for example employing a RACE reaction, at this stage the DNA can be sent for sequence analysis and cognate barcodes can be read;
3) the duplex probe molecule is then treated with RNAse to remove mRNA;
4) anneal primers to the 3′ tails of the extended probes (from step 2 above) and extend using a polymerase in the 5′ direction;
5) closing of nicks to form a fully double stranded DNA molecule or just transform into a host cell, such as E. coli.

This process is summarised in FIG. 10.

Step 1 Capture Cognate Pair mRNA

This step is relatively passive, doesn't require addition of enzymes or reagents. The process relies predominantly on an appropriate concentration of the duplex probe molecule. If there is too much duplex probe molecule present then this increases the likelihood of a situation where each duplex only captures mRNA on one of the probes in the molecule and not both. Too little duplex and there may not be a high enough concentration of the duplex probe molecule for the downstream reactions.

A tube based method involves pipetting the duplex probe molecule directly into the tube at an appropriate amount to give the desired concentration.

In a droplet microfluidics based method then the duplex probe molecule can be pre-loaded at the appropriate amount into the droplets.

### Step 2(a) RT Extension

This step involves a standard Reverse Transcription enzyme. Kits suitable for use in this step include, for example Invitrogen Superscript III Reverse Transcription kit available at https://www.thermofisher.com/order/catalog/product/18080093 First-Strand cDNA Synthesis

The following 20 μl reaction volume can be used for 10 pg to 5 ng of total RNA or 10 pg to 500 ng of mRNA.
1. Add the following components to a nuclease-free microcentrifuge tube:
   1 μl of oligo(dT)20 (50 μM); or 200 to 500 ng of oligo(dT)12-18; or
   50 to 250 ng of random primers; or 2 pmol of gene-specific primer 10 pg to 5 ng total RNA or 10 pg to 500 ng mRNA;
   1 μl 10 mM dNTP Mix (10 mM each dATP, dGTP, dCTP and dTTP at neutral pH); and Sterile, distilled water to 13 μl.
2. Heat mixture to 65° C. for 5 minutes and incubate on ice for at least 1 minute.
3. Collect the contents of the tube by brief centrifugation and add:
   4 μl 5× First-Strand Buffer; 1 μl 0.1 M DTT;
   1 μl RNaseOUT™ Recombinant RNase Inhibitor (Cat. no. 10777-019, 40 units/μl). Note: When using less than 50 ng of starting RNA, the addition of RNaseOUT™ is essential.
   1 μl of SuperScript™ III RT (200 units/μl)*
   *If generating cDNA longer than 5 kb at temperatures above 50° C. using a gene-specific primer or oligo(dT)20, the amount of SuperScript™ III RT may be raised to 400 U (2 μl) to increase yield.
4. Mix by pipetting gently up and down. If using random primers, incubate tube at 25° C. for 5 minutes.
5. Incubate at 50° C. for 30 to 60 minutes. Increase the reaction temperature to 55° C. for gene-specific primer. Reaction temperature may also be increased to 55° C. for difficult templates or templates with high secondary structure.
6. Inactivate the reaction by heating at 70° C. for 15 minutes.

### Step 2(b)

At this stage the samples can now be handled en masse as the barcodes have been physically added to the V-regions. This would then allow downstream PCR (and cloning if necessary) for the DNA to be made double stranded and amplified so that we can send for next generation sequencing.

### Step 3 RNAse Treatment (RNAse H)

This step is optional but may be included to degrade away the original messenger RNA and any other RNA that could contaminate downstream reactions. Amplification of some PCR targets (those >1 kb) may require the removal of RNA complementary to the cDNA. To remove RNA complementary to the cDNA, add 1 μl (2 units) of E. coli RNase H and incubate at 37° C. for 20 minutes.

### Step 4 Anneal and Extend Using Reverse Primers

Depending on which extension has been used in step 2 (RACE or just standard RT) they the appropriate primer sets are employed to anneal to the extended 3′ tails. Once the primers are place then a DNA polymerase is employed to make the complementary DNA strand and provide fully double stranded constructs as shown at the bottom of FIG. 10. They are not completely double stranded as there will still be the ‘nicks’ left over which need to be closed, for example with ligase.

Step 5 Blunt Ligation and Subsequent Transformation of E. coli.

Ligate the double stranded constructs using blunt ended cloning into appropriately cut blunt vectors by adding T4 DNA ligase (which close the ‘nicks’) and then transform E. coli either by electroporation or alternative methods such as heat shock methods.

Cloning into Vectors and Prepping DNA for Next Generation Sequencing

Adding 1 million duplex probe molecule (each with an individual barcode) into each tube or microfluidic droplet then this generates each antibody sequence barcoded (cognate) with 1 million different barcodes. Bursting the microfluidic droplets open, for example to combine the contents of 100,000 droplets, results in 1 million barcoded antibody sequences×100,000 which is 1×10¹¹ molecules of DNA to process and handle.

Next generation processing can handle 1 million sequences thus it may not be possible using next generation sequencing alone to guarantee that the corresponding VH and VL sequences sharing the same barcodes will be analysed. For some applications this may be acceptable.

However, by cloning (i.e. selecting out for further study) only 1 million sequences at the double stranded stage then the next generation sequencing provides data for all the antibodies and there will only ever be 1 million barcodes which will be matched.

### Example 8 mRNA Capture Using Long Constant Region Specific Primers

We have shown capture of mRNA using the long primers in isolation (prior to duplex construction).

These long primers anneal to mRNA as evidenced by cDNA product formation.
1. mRNA was extracted from a vial of rabbit bone marrow using QIAGEN RNeasy miniprep kit. https://www.qiagen.com/gb/resources/resourcedetail?id=Oe32fbbl-c307-4603-ac81-a5e98490ed23&lang=en
2. Conditions of mRNA capture by reverse transcription:
Oligo-dT (used as a control primer)
Gene specific primers
   Heavy: CH-POC-long3: Kappa: CK-POC long
Reverse Transcription polymerase chain reaction (RT-PCR) was performed using SuperScript IV
First-Strand SSIV protocol according to manufacturer's instructions (Thermo).

Reaction Mixture:
10.5 μl sterilised distilled water; —5 ng total RNA; —1 μl dNTPs (10 mM)
1 μl Oligo-dT (50 μM) OR 1 μl gene specific primer (2 μM)
   Anneal at 65° C. for 5 minutes.

Add Following Constituents to the Annealed Primer Mix:
4 μl SS IV buffer (5×); —1 μl DTT (100 mM); —1 μl Ribonuclease inhibitor:
1 μl Reverse transcriptase (200 U/μL)
   Extension at 50° C. for 10 minutes
   Denature at 80° C. 10 minutes)
3. PCR using KOD Master mix (Merck Millipore).
Four different experimental conditions were performed (A-D) using either oligo-dT-derived cDNA or gene-specific primer (GSP)-derived cDNA.

PCR Conditions for Testing Gene Specific Primers and mRNA Quality

| Condition | Primers |
|---|---|
| A | #3, #7 |
| B | #1, #2, #6 |
| C | #3, #8, #9 |
| D | #1, #2, #10 |

PCR Mixture:
20 μl sterilised distilled water; —2 μl cDNA template (from step 2); 1.5 μl Forward primer (10 μM); 1.5 μl Reverse primer (10 μM); and—25 μl KOD MasterMix

PCR Cycling Conditions:
1. KOD Polymerase activation 95° C. for 2 minutes
2. Denature step performed at 95° C. for 20 seconds
3. Anneal step performed above 55° C. for 10 seconds
4. Extension step performed at 70° C. for 15 seconds
   Repeat steps 2 to 4 for 30 cycles

| Primer | | | |
|---|---|---|---|
| # | Primer Name | Sequence (5′-3′) | Type |
| 1 | Rab Kappa 1ry | CGCGCGAAGCTTCGAAGCCACCATGGACAYGAG | Forward |
| | For1AY | GGCCCCCACTCAG | |
| | | SEQ ID NO: 19 | |
| 2 | Rab Kappa 1ry | CGCGCGAAGCTTCGAAGCCACCATGAACAYGAG | Forward |
| | For2AY | GGCCCCCACTCAG | |
| | | SEQ ID NO: 20 | |
| 3 | RbVH 1ry 2015C2 | AAGCTTACGCTCACCATGGAGACTGGGCTGCGCT | Forward |
| | | GG SEQ ID NO: 21 | |
| 4 | H Rev for BC | CCTACTGTCCTCATCTTCCCGCAGGGCGCAGCTC | Reverse |
| | PCR | GGAC SEQ ID NO: 22 | |
| 5 | K Rev for BC | CCATCAGTCTTCCCACGGGACGCGCCCGTGTGCA | Reverse |
| | PCR | G SEQ ID NO: 23 | |
| 6 | CK-POC long | AGCTATAGCTGAGGATGCAGTGCTGCCAGTCCGA | Reverse |
| | | GCTGCGCCCTGCGGGAAGATGAGGACAGTAGGT | |
| | | GCAACTGG SEQ ID NO: 24 | |
| 7 | CH-POC-1ong3 | ATATCGCCTCAGCCGATGCCTGTAGCCTGCACAC | Reverse |
| | | GGGCGCGTCCCGTGGGAAGACTGATGGAGCCTTA | |
| | | GGTTGCC SEQ ID NO: 25 | |
| 8 | Rab Heavy 1ry | CAGGTCACGGTCACTGGCTC SEQ ID NO: 26 | Reverse |
| | Rev1C | | |
| 9 | Rab Heavy 1ry | GCCYTCTAGATGMMTGCT SEQ ID NO: 27 | Reverse |
| | Rev2 | | |
| 10 | Rab Kappa 1ry | CGCCACACACACACGATGGTGACTG | Reverse |
| | Rev2 | SEQ ID NO: 28 | |

See FIG. 16, which shows capturing mRNA using oligo-dT (50 PIM) or using the constant region-specific primer—heavy or kappa long primer (2 μM). Synthesised cDNA was used in a standard PCR reaction as using primers detailed in Example 2.

### Example 9 Using the Constructed in Example 2 to Amplify Heavy and Light Chain Variable and Constant-Region Containing IgG Transcripts

1. Reverse Transcription reaction using SuperScript IV—First-Strand SSIV (protocol at https://www.thermofisher.com/order/catalog/product/18091050) to capture heavy and light chain mRNA.

Reaction Mixture:
10.5 μl sterilised distilled water;—5 ng RNA:—1 μl dNTPs (10 mM)
1 μl Oligo dT (50 μM) OR 0.5 μM barcode duplex (constructed in Section 2)
   Anneal at 65° C. for 5 minutes.

The Following Constituents were Added to the Annealed Primer Mix:
4 μl Superscript IV buffer (5×);—1 μl DTT (100 mM);—1 μl Ribonuclease inhibitor
1 μl Reverse transcriptase (200 U/μL)
   Extension at 50° C. for 10 minutes
   Denature at 80° C. 10 minutes)
2. Presumed cDNA product from reverse transcription reaction was used as template DNA in PCR.

Reaction Mix:
12.5 μl KOD Mastermix; 10.5 μl sterilised distilled water; 0.75 μl total forward primer;
0.75 μl total reverse primer
0.5 μl cDNA

PCR Cycling Conditions:
1. Polymerase activation 95° C. for 2 minutes
2. Denature step performed at 95° C. for 20 seconds
3. Anneal step performed above 55° C. for 10 seconds
4. Extension step performed at 70° C. for 15 seconds
   Primer conditions used in amplification of heavy and light variable regions captured either by POC Duplex molecule of oligo-dT. Conditions kept the same for second round of PCR

| Condition | Primers (see above) |
|---|---|
| H Long | #3, #7 |
| K Long | #1, #2, #6 |
| H Design | #3, #4 |
| K Design | #1, #2, #5 |
| Both fwd | #1, #2, #3 |

3. Product purification using QIAGEN PCR Purification Kit.
4. Repeat PCR with same conditions, replacing 0.5 μl of cDNA with 1 μl of primary PCR product See FIG. 19, which shows PCR products after 2 rounds of PCR amplification using primer conditions in the table above. BCD captured: Using POC Duplex molecule constructed in Section 2. There was no product observed in from the use of only forward primers to get joined heavy and light chain variable regions from same duplex molecule

### Example 10 PCR and Cloning

The PCR products generated in the experiments described in Example 8 were sub-cloned using the CloneJET system (Thermo) and sub-cloned inserts were sequences to determine the captured mRNA transcripts' sequence using the primers supplied with the system.

Variable domains were captured and sequenced (sequences not disclosed herein).

A band observed at 1000 base pairs is an indication that both parts of the probe are capable of binding mRNA. The conditions for capture require optimisation.

1. A duplex probe molecule comprising:
i) a double-stranded-core (the core) comprising a first polynucleotide strand (first-strand) and a second polynucleotide strand (second-strand), wherein the first and second strands are complementary to each other,
ii) a single stranded first polynucleotide probe (first probe) sequence extending in the 5′ to 3′ direction from the first-strand of the core; and
iii) single stranded second polynucleotide probe (second probe) sequence extending in the 5′ to 3′ direction from the second strand of the core;
wherein the first and second probes extend outwards from the double stranded core in opposing directions on different polynucleotide strands and each terminate in a 3′ end.

2. A duplex probe molecule according to claim 1 comprising a barcoding region.

3. A duplex probe molecule according to claim 1 where a barcoding region is located between the first probe and the first-strand of the core.

4. A duplex probe molecule according to claim 1, where a barcoding region is located between the second probe and the second-strand of the core.

5. A duplex probe molecule according to claim 1, wherein the first-strand of the core comprises a barcode.

6. A duplex probe molecule according to claim 1, wherein the second-strand of the core comprises a barcode.

7. A duplex probe molecule according to claim 1, wherein the first strand comprises a first and third primer annealing site.

8. A duplex probe molecule according to claim 1, wherein the second strand comprises second and fourth primer annealing site.

9. A duplex probe molecule according to claim 1 comprising:
a double stranded core (the core) of a first polynucleotide strand (first-strand) and a second polynucleotide strand (second-strand), wherein the first-strand comprises a barcode sequence flanked by a first and a second primer annealing site and the second-strand comprises a barcoding sequence flanked by a third and a fourth primer annealing site;
a single stranded first polynucleotide probe sequence (first probe) extending in the 5′ to 3′ direction from the first-strand of the core; and
a single stranded second polynucleotide probe sequence (second probe) extending in the 5′ to 3′ direction from the second-strand of the core,
such that the first and second probes extend outwards from the double stranded core in opposing directions on different polynucleotide strands and each terminate in a 3′ end.

10. The duplex probe molecule according to claim 2, wherein the double stranded core comprises a barcode having 6 to 100 base pairs.

11. The duplex probe molecule according claim 1, wherein the first and/or second probe sequences encode one or more antibody regions.

12. The duplex probe molecule according to claim 11, wherein the first polynucleotide probe sequence encodes a heavy chain constant region and the second polynucleotide probe sequence encodes a light chain constant region or vice versa.

13. A host cell comprising a duplex probe molecule according to claim 1.

14. A kit comprising one or more duplex probe molecules according to claim 1 and reagents and/or instructions for use.

15. A method of preparing a duplex probe molecule according to claim 1, comprising the steps of:
a) providing a strand-one comprising in the 3′ to 5′ direction, a first polynucleotide probe, a first primer annealing site, a barcoding sequence and a restriction site, and:
i. annealing to strand-one a first primer specific to the first primer annealing site, and
ii. employing a polymerase to synthesise the complementary polynucleotide sequence from the first primer along the length of strand-one in the 5′ to 3′ direction to provide a double stranded barcode region, and
b) providing a strand-two comprising in the 3′ to 5′ direction, the second polynucleotide probe, a second primer annealing site, a barcoding sequence and a restriction, and annealing to strand-two:
i. a second primer specific to the second primer annealing site, and
ii. employing a polymerase to synthesise the complementary polynucleotide sequence from the second primer along the length of strand-two in the 5′ to 3′ direction to provide a double stranded barcode region and,
c) cutting a double stranded part of strand-one and strand-two with a restriction enzyme specific to the restriction site encoded therein, in the same or separate reactions; and
d) ligating the ends of strand-one and strand-two obtained from step c) to form a duplex probe molecule comprising a double stranded core made up of the double stranded region from strand-one ligated to the double stranded 5′ region from strand-two, such that the first probe sequences and second probe sequence each extend as a single strand from the relevant double stranded region in opposing directions and each single strand terminates in a 3′ end.

16. The method according claim 15, wherein the sticky ends have a sense strand that is non-palindromic to a sticky end in the corresponding antisense strand of polynucleotide.

17. The method according to claim 15 wherein the restriction site is one that is cut using an enzyme selected from the group consisting of: AciI, AcuI, AlwI, BaeI, BbsI, BbvCI, BbvI, BccI, BceAI, BcgI, BciVI, BcoDI, BfuAI, BmrI, BpmI, Bpul0I, BpuEI, BsaI(1), BsaI-HF®, BsaXI, BseRI, BseYI, BsgI, BsmAI, BsmBI, BsmFI, BsmI, BspCNI, BspMI, BspQI, BsrDI, BsrI, BssSαI, BssSI, BtgZI, BtsaI, BtsCI, BtsI, BtsI MutI, CspCI, Earl, EciI, EciI, EcoP15I, FauI, FokI, FspEI, HgaI, HphI, HpvAV, I-CeuI, I-SceI, LpnPI, MboII, MmeI, Mn1I, MspJI, Nb.BbvCI, Nb. BsmII, Nb.BsrDI, Nb.BtsI, NmeAIII, Nt.AlwI, Nt.BbvCI, Nt.BsmAI, Nt.BspQI, Nt.CviPII, PI-PspI, PI-SceI, PleI, SapI and SfaNI.

18. The method according to claim 17, wherein the restriction enzyme is BbvCI, in strand-one and/or strand-two.

19. The method according to claim 15, wherein the same restriction enzyme is used to cut both strand one and strand two.

20. The method according to claim 15, wherein the strand-one and/or strand-two molecules further comprise one or more base pairs upstream (in the 5′ direction) of the restriction site.


SEQUENCE LISTING (29 sequences)

SEQ ID NO: 1 - DNA, 76 bp, Artificial Sequence
  21889 CH-BC long
  misc_feature (15)..(28): n represents a base which represents any one of g, a, t or c
catatcgcctcagcnnnnnnnnnnnnnnctgcacacgggcgcgtcccgtgggaagactgacggacgccttaggttg

SEQ ID NO: 2 - DNA, 75 bp, Artificial Sequence
  21888 CK-BC long
  misc_feature (15)..(28): n represents a base which represents any one of g, a, t or c
agctatagctgaggnnnnnnnnnnnnnngtccgagctgcgccctgcgggaagatgaggacagtaggtgcaactgg

SEQ ID NO: 3 - DNA, 19 bp, Artificial Sequence
  CH-BC short
gggacgcgcccgtgtgcag

SEQ ID NO: 4 - DNA, 19 bp, Artificial Sequence
  CK-BC short
cgcagggcgcagctcggac

SEQ ID NO: 5 - DNA, 19 bp, Artificial Sequence
  CH-BC long fragment
cgcagggcgcagctcggac

SEQ ID NO: 6 - DNA, 19 bp, Artificial Sequence
  CK-BC long fragment
gggacgcgcccgtgtgcag

SEQ ID NO: 7 - DNA, 10 bp, Artificial Sequence
  Restriction Site
  misc_feature (3)..(6): n represents a base which represents any one of g, a, t or c
  misc_feature (10)..(10): y is t/u or c
acnnnngtay

SEQ ID NO: 8 - DNA, 12 bp, Artificial Sequence
  Restriction Site
  misc_feature (4)..(8): n represents a base which represents any one of g, a, t or c
caannnnngtgg

SEQ ID NO: 9 - DNA, 12 bp, Artificial Sequence
  Restriction Site
  misc_feature (4)..(4): n is a, c, g, or t
  misc_feature (5)..(9): n represents a base which represents any one of g, a, t or c
cgannnnnntgc

SEQ ID NO: 10 - DNA, 27 bp, Artificial Sequence
  Restriction Site
taactataacggtcctaaggtagcgaa

SEQ ID NO: 11 - DNA, 18 bp, Artificial Sequence
  Restriction Site
tagggataacagggtaat

SEQ ID NO: 12 - DNA, 11 bp, Artificial Sequence
  Restriction Site
  misc_feature (3)..(7): n represents a base which represents any one of g, a, t or c
acnnnnnctcc

SEQ ID NO: 13 - DNA, 30 bp, Artificial Sequence
  Restriction Site
tggcaaacagctattatgggtattatgggt

SEQ ID NO: 14 - DNA, 30 bp, Artificial Sequence
  Restriction Site
atctatgtcgggtgcggagaaagaggtaat

SEQ ID NO: 15 - DNA, 75 bp, Artificial Sequence
  Primer
agctatagctgaggatgcagtgctgccagtccgagctgcgccctgcgggaagatgaggacagtaggtgcaactgg

SEQ ID NO: 16 - DNA, 74 bp, Artificial Sequence
  Primer
atatcgcctcagccgatgcctgtagcctgcacacgggcgcgtcccgtgggaagactgatggagccttaggttgc

SEQ ID NO: 17 - protein, 48 aa, Artificial Sequence
  Rabbit Constant Region
  misc_feature (17)..(17): Xaa can be any naturally occurring amino acid
RDPVAPSVLLFPPSKEXDPVAPTVLIFPPAADGQPKAPSVFPLAPCCG

SEQ ID NO: 18 - DNA, 144 bp, Artificial Sequence
  Rabbit Constant Region
  misc_feature (49)..(49): n is a, c, g, or t
cgtgatccagttgcgccttctgtcctcctcttcccaccatctaaggagngtgatccagttgcacctactgtcctcatcttcccaccagctgctgatgggcaacctaaggctccgtcagtcttcccactggccccctgctgcggg

SEQ ID NO: 19 - DNA, 46 bp, Artificial Sequence
  Primer
cgcgcgaagcttcgaagccaccatggacaygagggcccccactcag

SEQ ID NO: 20 - DNA, 46 bp, Artificial Sequence
  Primer
  misc_feature (30)..(30): y is t/u or c
cgcgcgaagcttcgaagccaccatgaacaygagggcccccactcag

SEQ ID NO: 21 - DNA, 36 bp, Artificial Sequence
  Primer
aagcttacgctcaccatggagactgggctgcgctgg

SEQ ID NO: 22 - DNA, 38 bp, Artificial Sequence
  Primer
cctactgtcctcatcttcccgcagggcgcagctcggac

SEQ ID NO: 23 - DNA, 35 bp, Artificial Sequence
  Primer
ccatcagtcttcccacgggacgcgcccgtgtgcag

SEQ ID NO: 24 - DNA, 75 bp, Artificial Sequence; identical to SEQ ID NO: 15
  Primer

SEQ ID NO: 25 - DNA, 75 bp, Artificial Sequence
  Primer
atatcgcctcagccgatgcctgtagcctgcacacgggcgcgtcccgtgggaagactgatggagccttaggttgcc

SEQ ID NO: 26 - DNA, 20 bp, Artificial Sequence
  Primer
caggtcacggtcactggctc

SEQ ID NO: 27 - DNA, 18 bp, Artificial Sequence
  Primer
  misc_feature (4)..(4): y is t/u or c
  misc_feature (13)..(14): m is a or c
gccytctagatgmmtgct

SEQ ID NO: 28 - DNA, 25 bp, Artificial Sequence
  Primer
cgccacacacacacgatggtgactg

SEQ ID NO: 29 - DNA, 10 bp, Artificial Sequence
  Cleavage site
  misc_feature (7)..(10): n represents a base which represents any one of g, a, t or c
ccagagnnnn